SEQUENCE LISTING (45 sequences)

SEQ ID NO: 1 - DNA, 2337 bp, Homo sapiens
acattctgccctgatttccggaacctggaagcctaggcaggcagtggggaactctgactcgcctgtgctctggagcttgatccgaaagcttccacagtgaggactgctccgtgggggtaagagagcaccaggcactgaggcctgggagttccacagaccaacacccctgctcctggcggctcccacccgggacttagaccctcaggtccctaatatcccggaggtgctctcaatcagaaaggtcctgctccgcttcgcagtggaatggaacggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaagtgctgcaacctaaaccagcaattacacccaagctcgttggggcctaagccagtaccgacctggtagaaaaagcaaccacgaagctagagagagagccagaggagggaagagagcgccagacgaaggtgaaagcgaaccacgcagagaaatgcaggcaagggagcaaggcggcagttcccggaacaaacgtggcagagggcaagacgggcactcacagacagaggtttatgtatttttattttttaaaatctgatttggtgttccatgaggaaaagggaaaatctagggaacgggagtacagagagaataatccgggtcctagctcgccacatgaacgcccagagaacgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagaccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaaggggttgggagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggacttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagacacagtggcaccgcttttctaactggcagcacagagcaactctataatatgcttatattaggtctagaagaatgcatcttgagacacatgggtaacctaattatataatgcttgttccatacaggagtgattatgcagtgggaccctgctgcaaacgggactttgcactctaaatatagaccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaatgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttctgtgcactggttttatatgccttatggagtatatactcacatgtagctaaatagactcaggactgcacattccttgtgtaggttgtgtgtgtgtggtggttttatgcataaataaagttttacatgtggtgaaaaaa

SEQ ID NO: 2 - DNA, 4 bp, Artificial Sequence
  Description of Artificial Sequence Synthetic oligonucleotide
aggt

SEQ ID NO: 3 - DNA, 4 bp, Artificial Sequence
  Description of Artificial Sequence Synthetic oligonucleotide
agct

SEQ ID NO: 4 - DNA, 23 bp, Artificial Sequence
  Description of Artificial Sequence Synthetic primer
ggtagaaaaagcaaccacgaagc

SEQ ID NO: 5 - DNA, 25 bp, Artificial Sequence
  Description of Artificial Sequence Synthetic primer
acataaacctctgtctgtgagtgcc

SEQ ID NO: 6 - DNA, 22 bp, Artificial Sequence
  Description of Artificial Sequence Synthetic primer
ccgggaaactgtggcgtgatgg

SEQ ID NO: 7 - DNA, 25 bp, Artificial Sequence
  Description of Artificial Sequence Synthetic primer
aggtggaggagtgggtgtcgctgtt

SEQ ID NO: 8 - DNA, 21 bp, Artificial Sequence
  Description of Artificial Sequence Synthetic primer
gccacattctctactcggtga

SEQ ID NO: 9 - DNA, 20 bp, Artificial Sequence
  Description of Artificial Sequence Synthetic primer
ccaagcctgagacctactgc

SEQ ID NO: 10 - DNA, 20 bp, Artificial Sequence
  Description of Artificial Sequence Synthetic primer
tgacctgtctgcaaatgctc

SEQ ID NO: 11 - DNA, 19 bp, Artificial Sequence
  Description of Artificial Sequence Synthetic primer
cagaccctggttgcttcaa

SEQ ID NO: 12 - DNA, 19 bp, Artificial Sequence
  Description of Artificial Sequence Synthetic primer
ctctgcttctcgctcctcc

SEQ ID NO: 13 - DNA, 20 bp, Artificial Sequence
  Description of Artificial Sequence Synthetic primer
tctgtgaagttcccgatcaa

SEQ ID NO: 14 - DNA, 20 bp, Artificial Sequence
  Description of Artificial Sequence Synthetic primer
ggacacttcactttgctgcc

SEQ ID NO: 15 - DNA, 20 bp, Artificial Sequence
  Description of Artificial Sequence Synthetic primer
tagtgcctggggttcaacat

SEQ ID NO: 16 - DNA, 18 bp, Artificial Sequence
  Description of Artificial Sequence Synthetic primer
tcttttggggcagaaaac

SEQ ID NO: 17 - DNA, 15 bp, Artificial Sequence
  Description of Artificial Sequence Synthetic primer
aagatggcgaggagg

SEQ ID NO: 18 - DNA, 20 bp, Artificial Sequence
  Description of Artificial Sequence Synthetic primer
cccgtctacactgtgtccct

SEQ ID NO: 19 - DNA, 20 bp, Artificial Sequence
  Description of Artificial Sequence Synthetic primer
ggagtacacgacctcaccgt

SEQ ID NO: 20 - DNA, 20 bp, Artificial Sequence
  Description of Artificial Sequence Synthetic primer
aggtgtgtttgaccggagac

SEQ ID NO: 21 - DNA, 20 bp, Artificial Sequence
  Description of Artificial Sequence Synthetic primer
tccctatttcttcaccagcg

SEQ ID NO: 22 - DNA, 20 bp, Artificial Sequence
  Description of Artificial Sequence Synthetic primer
acctgacttccagcacgagt

SEQ ID NO: 23 - DNA, 20 bp, Artificial Sequence
  Description of Artificial Sequence Synthetic primer
ccaactcctttcttcccctc

SEQ ID NO: 24 - DNA, 20 bp, Artificial Sequence
  Description of Artificial Sequence Synthetic primer
gctgaggcgctttaatgaac

SEQ ID NO: 25 - DNA, 20 bp, Artificial Sequence
  Description of Artificial Sequence Synthetic primer
ggtcccagaaactctgacca

SEQ ID NO: 26 - DNA, 20 bp, Artificial Sequence
  Description of Artificial Sequence Synthetic primer
tctccaacttctgtccgagg

SEQ ID NO: 27 - DNA, 20 bp, Artificial Sequence
  Description of Artificial Sequence Synthetic primer
cacgagtttgatgccagaga

SEQ ID NO: 28 - DNA, 20 bp, Artificial Sequence
  Description of Artificial Sequence Synthetic primer
atatgacaaacacggcccat

SEQ ID NO: 29 - DNA, 20 bp, Artificial Sequence
  Description of Artificial Sequence Synthetic primer
ggtggttaacttggggaaca

SEQ ID NO: 30 - DNA, 20 bp, Artificial Sequence
  Description of Artificial Sequence Synthetic primer
accaggctctgttctgttcg

SEQ ID NO: 31 - DNA, 20 bp, Artificial Sequence
  Description of Artificial Sequence Synthetic primer
tcttgggtcataggggtctg

SEQ ID NO: 32 - DNA, 20 bp, Artificial Sequence
  Description of Artificial Sequence Synthetic primer
agaccggcaatttgcttcta

SEQ ID NO: 33 - DNA, 20 bp, Artificial Sequence
  Description of Artificial Sequence Synthetic primer
tctggggcatggtcatttat

SEQ ID NO: 34 - RNA, 21 nt, Artificial Sequence
  Description of Artificial Sequence Synthetic oligonucleotide
gaacgggaguacagagagauu

SEQ ID NO: 35 - RNA, 21 nt, Artificial Sequence
  Description of Artificial Sequence Synthetic oligonucleotide
ccacaugaacgcccagagauu

SEQ ID NO: 36 - RNA, 21 nt, Artificial Sequence
  Description of Artificial Sequence Synthetic oligonucleotide
uaacaagaccagagagcuguu

SEQ ID NO: 37 - DNA, 21 bp, Artificial Sequence
  Description of Artificial Sequence Synthetic oligonucleotide
cuacaacagccacaacguctt

SEQ ID NO: 38 - DNA, 8 bp, Artificial Sequence
  Description of Artificial Sequence Synthetic oligonucleotide
agggacag

SEQ ID NO: 39 - DNA, 5 bp, Artificial Sequence
  Description of Artificial Sequence Synthetic oligonucleotide
ccagc

SEQ ID NO: 40 - DNA, 5 bp, Artificial Sequence
  Description of Artificial Sequence Synthetic oligonucleotide
ccagg

SEQ ID NO: 41 - DNA, 12 bp, Artificial Sequence
  Description of Artificial Sequence Synthetic oligonucleotide
atggacagcgcc

SEQ ID NO: 42 - DNA, 5 bp, Artificial Sequence
  Description of Artificial Sequence Synthetic oligonucleotide
ccagc

SEQ ID NO: 43 - DNA, 5 bp, Artificial Sequence
  Description of Artificial Sequence Synthetic oligonucleotide
ccagg

SEQ ID NO: 44 - DNA, 23 bp, Artificial Sequence
  Description of Artificial Sequence Synthetic oligonucleotide
aannnnnnnnnnnnnnnnnnntt

SEQ ID NO: 45 - DNA, 4 bp, Artificial Sequence
  Description of Artificial Sequence Synthetic oligonucleotide
tcga

(19) United States
(12) Patent Application Publication
(10) Pub. No.: US 2012/0004278 A1
(43) Pub. Date: Jan. 5, 2012

(54) LINC RNAS IN CANCER DIAGNOSIS AND TREATMENT

(75) Inventors: Howard Y. Chang, Stanford, CA (US); Rajnish A. Gupta, Sunnyvale, CA (US)

(73) Assignee: THE BOARD OF TRUSTEES OF THE LELAND STANFORD JUNIOR UNIVERSITY, PALO ALTO, CA (US)

(21) Appl. No.: 13/163,228

(22) Filed: Jun. 17, 2011

Related U.S. Application Data
(60) Provisional application No. 61/356,166, filed on Jun. 18, 2010.

(51) Int. Cl. A61K 31/7088; A61K 31/7105; C40B 30/04; A61P 35/00; C12Q 1/68; C40B 60/12; C12M 1/34; A61P 35/04; A61K 31/713; C12N 5/09

(52) U.S. Cl. 514/44 A; 506/9; 435/375; 435/6.12; 435/6.11; 506/39; 435/287.2

(57) ABSTRACT

Long non-coding RNAs (lincRNAs), a relatively recently recognized class of widely transcribed genes, are thought to affect chromatin state and epigenetic regulation, but their mechanisms of action and potential roles in human disease are poorly understood. The present invention shows that long non-coding RNAs in the human HOX loci are systematically dysregulated during breast cancer progression, and that expression levels of the lincRNA termed HOTAIR can predict cancer metastasis. Elevated levels of HOTAIR can lead to altered patterns of Polycomb binding to the genome. These findings indicate that lincRNAs have active roles in modulating the cancer epigenome and may be important targets for cancer diagnosis and therapy.

### CROSS REFERENCE TO RELATED APPLICATIONS

This application claims benefit under 35 U.S.C. 119(e) of U.S. Provisional Patent Application Ser. No. 61/356,166 filed on Jun. 18, 2010, the contents of which is incorporated herein in its entity by reference.

### GOVERNMENT SUPPORT

This invention was made with U.S. government support under Grant No. R01-CA118750 awarded by the NIH National Cancer Institute. The government has certain rights in the invention.

### FIELD OF THE INVENTION

The present invention relates to epigenetics and the cancer epigenome; and provides for compositions and methods to diagnose, predict prognosis, and identify therapeutic epigenetic targets in human cancers. More specifically, the misexpression of large intervening noncoding RNAs (lincRNAs) can reprogram the epigenetic states of cells, leading to cancer progression.

### BACKGROUND OF THE INVENTION

Cancer is a leading cause of disease-related death in the U.S. Worldwide, breast cancer is the fifth most common cause of cancer death. Over 200,000 American women were diagnosed with breast cancer in 2006, and about 40,000 women die annually from this disease. The incidence of breast cancer had been rising in American women for more than thirty years. Because the breast is composed of identical tissues in males and females, breast cancer also occurs in males, though less often. Breast cancer is not only a serious physical disease, but it is often an emotionally draining disease as well. In many cancers, protein-coding genes may be are misexpressed by several- to dozens-fold, but remain elusive by current detection and measurement techniques. Further, the epigenetic states of human cancers, such as chromatin modification of specific genes, are difficult to measure in patient samples. Thus, there remains a need for techniques to better determine cancer prognosis and treat this disease.

### SUMMARY OF THE INVENTION

Embodiments of the present invention provide for novel biomarkers, lincRNAs, that reprogram the epigenetic state of human cells and enable cancer progression. Specific lincRNAs, such as HOTAIR, can predict the prognosis of human cancer, and are themselves therapeutic targets. Additionally lincRNAs provide for downstream targets, e.g., HOTAIR LincRNA's downstream affect on PRC2, that are also useful in cancer prognosis and therapeutics. For example, PRC2 target genes, such as JAM2, PCDH10, PCDHB5, were transcriptionally repressed upon HOTAIR LincRNA expression and de-repressed upon concomitant PRC2 depletion. Hence, specific reagents that inhibit lincRNAs and their downstream functions, and decrease tumorigenicity, can be used in cancer therapy. Further, lincRNAs can be used in creating models of human cancers. Cell lines that harbor specific lincRNA alterations can be used to screen compounds that block the actions of lincRNAs. Thus, the present invention provides for further understanding and treatment of cancers that arise from genetic as well as epigenetic abnormalities.

Other embodiments provide for detecting lincRNAs by standard molecular methods; or for quantifying increased or decreased expression of lincRNAs by standard molecular methods. Such methods include any method of nucleic acid detection, for example in situ hybridization detection of HOTAIR LincRNA using antisense DNA or cRNA oligonucleotide probes, ultra-high throughput sequencing, Nanostring technology, microarrays, rolling circle amplification, proximity-mediated ligation, PCR, qRT-PCR ChIP, ChIP-qPCR or antibodies, or protein or nucleic acid measurements of any of the several members that comprise PRC2 gene set. Additionally, the use of cells and/or animal models harboring lincRNA alterations, as taught herein, allows development of detection agents, identifying antibodies, small molecule compounds, or RNA interference that further identify or target lincRNA pathways.

Unlike protein-coding genes that are misexpressed by several- to dozens-fold in many cancers, lincRNAs are misexpressed by thousands-fold, greatly facilitating their detection and measurement. Currently, the epigenetic states of human cancers, such as chromatin modification of specific genes, are difficult to measure in patient samples. Such epigenetic states can be identified, however, by measurement of lincRNA levels as described herein. Moreover, lincRNAs are prevalent epigenetic abnormalities in cancer that are shown herein to be therapeutic drug targets.

One aspect of the present invention relates to a method for the treatment of metastatic cancer in a subject comprising administering to a subject having metastatic cancer an effective amount of a RNAi inhibitor of HOTAIR lincRNA function and/or its expression from the HOTAIR gene. In some embodiments, the presence of metastatic cancer in the subject is indicated by high levels of HOTAIR lincRNA expression, for example as measured by the methods and systems as disclosed herein. In some embodiments, high levels of HOTAIR LincRNA is at least about 125-fold increased as compared to a reference HOTAIR LincRNA level. In some embodiments, high levels of HOTAIR LincRNA is between about 125-fold and 2000-fold, or greater than 2000-fold increased as compared to a reference HOTAIR LincRNA level.

One aspect of the present invention relates to a method for decreasing HOTAIR lincRNA level in a cancer cell, comprising contacting the cancer cell with a RNAi inhibitor of HOTAIR lincRNA function and/or a RNAi inhibitor of HOTAIR LincRNA expression from the HOTAIR gene. In some embodiments, a RNAi inhibitor of HOTAIR lincRNA function or its expression from the HOTAIR gene can be selected from the group consisting of siRNA, miRNA, stRNA, snRNA, and antisense nucleic acid, and in some embodiments, can be a siRNA which targets HOTAIR LincRNA and/or its expression from the HOTAIR gene.

One aspect of the present invention relates to a method for detecting a metastatic cancer in a subject, comprising; (a) contacting a biological sample from the subject with at least one nucleic acid binding probe to measure the level of HOTAIR lincRNA in the biological sample; (b) comparing the level of HOTAIR lincRNA in the biological sample to a reference level of HOTAIR lincRNA from a biological sample from a healthy population, wherein an increased level of HOTAIR lincRNA in the biological sample from the subject compared to the reference level of HOTAIR lincRNA indicates likelihood of the subject having a metastatic cancer.

Another aspect of the present invention relates to a method for treating a metastatic cancer in a subject, comprising; (a) contacting a biological sample from the subject with at least one nucleic acid binding probe to measure the level of HOTAIR lincRNA in the biological sample; (b) comparing the level of HOTAIR lincRNA in the biological sample to a reference level of HOTAIR lincRNA from a biological sample from a healthy population, wherein an increased level of HOTAIR lincRNA in the biological sample from the subject compared to the reference level of HOTAIR lincRNA indicates likelihood of the subject having a metastatic cancer; and (c) administering a RNAi inhibitor of HOTAIR LincRNA and/or its expression from the HOTAIR gene in an effective amount to inhibit cancer metastasis in the subject.

Another aspect of the present invention relates to a method for treating a metastatic cancer in a subject, comprising; (a) contacting a biological sample from the subject with at least one nucleic acid binding probe to measure the level of HOTAIR lincRNA in the biological sample; (b) comparing the level of HOTAIR lincRNA in the biological sample to a reference level of HOTAIR lincRNA from a biological sample from a healthy population, wherein an increased level of HOTAIR lincRNA in the biological sample from the subject compared to the reference level of HOTAIR lincRNA indicates likelihood of the subject having a metastatic cancer; and (c) administering an agent which inhibits the function of PRC2 in an effective amount to inhibit cancer metastasis in the subject.

In some embodiments, an agent which inhibits the function PRC2 is a small molecule inhibitor of PRC2. In some embodiments, an agent which inhibits the function PRC2 is an agent which inhibits the interaction of HOTAIR lincRNA with PRC2.

In all aspects as disclosed herein, metastatic cancer is breast cancer.

Another aspect of the present invention relates to an assay for measuring the level of HOTAIR lincRNA in a biological sample from a subject; comprising at least one agent that specifically binds to HOTAIR lincRNA, wherein binding of the agent to HOTAIR lincRNA results in a detectable signal. In some embodiments, the assay is automated, e.g., carried out by a computerized system.

Another aspect of the present invention relates to a device comprising an assay for measuring the level of HOTAIR lincRNA in a biological sample from a subject, wherein the device comprises a solid support wherein the agent that specifically binds to HOTAIR lincRNA which deposited on the support. In some embodiments, the solid support is in the format of a dipstick, a test strip, a latex bead, a microsphere, or a multi-well plate. In some embodiments, the assay is automated, e.g., carried out by a computerized system.

Another aspect of the present invention relates to a device comprising; (a) a measuring assembly yielding a detectable signal from an assay indicating the level of HOTAIR lincRNA from a biological sample from a subject; and (b) an output assembly for displaying an output content for the user. In some embodiments, the assay is automated, e.g., carried out by a computerized system.

Another aspect of the present invention relates to a method for inhibiting invasive growth of a cancer cell that misexpresses HOTAIR comprising contacting said cell with a RNAi inhibitor that decreases HOTAIR lincRNA in said cell.

Another aspect of the present invention relates to a method of modulating histone H3 lysine 27 methylation in a cell comprising contacting that cell with a RNAi inhibitor of HOTAIR lincRNA.

### DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1F show that HOX lincRNAs are systematically dysregulated in breast carcinoma and have prognostic value for metastasis and survival. FIG. 1A shows a heat map representing unsupervised hierarchical clustering of expression values of a panel of primary and metastatic breast cancers relative to normal breast epithelial cells. An ultra-high density HOX tiling array (Rinn et al., 2007), was interrogated with either normal breast organoid RNA (Cy3 channel) or RNA derived from primary or metastatic breast tumors (Cy5 channel). Each column represents the indicated clinical sample. Each row indicates a transcribed region, either a HOX coding exon or HOX non-coding RNA (ncRNA). Expression values are depicted as a ratio relative to pooled normal and represented as a red-green color scale. FIG. 1B shows a higher resolution of subset (iii) identifying transcripts (including HOTAIR) that show higher relative expression in metastatic as compared to primary tumors and normal epithelia. FIG. 1C show quantitative reverse transcription (qRT)-PCR validation of the expression tiling array results measuring HOTAIR LincRNA abundance in a panel of normal breast epithelial-enriched organoids, primary breast tumors, and metastatic breast tumors. Metastatic tumors had at a minimum 125-fold higher level of HOTAIR LincRNA than normal breast epithelia. FIG. 1D show qRT-PCR analysis of HOTAIR LincRNA in 132 primary breast tumors (stage I or II). Approximately one third of primary breast tumors had >125 fold overexpression of HOTAIR LincRNA over normal (HOTAIR high, indicated in gray), while roughly two third of tumors did not (HOTAIR low, indicated in dark gray). FIG. 1E shows Kaplan-Meier curves for metastasis free survival and FIG. 1F shows Kaplan-Meier curves for metastasis overall survival of the same 132 primary breast tumors measured in the panel shown in FIG. 1D. Color versions of the drawings are available at Gupta et al., 464 Nature 1071-76 (2010).

FIGS. 2A-2D show that HOTAIR LincRNA promotes invasion of breast carcinoma cells. FIG. 2A shows the relative fold increase in matrix invasion in three breast carcinoma cell lines after enforced expression of HOTAIR LincRNA. Mean+s.d. are shown. FIG. 2B shows the matrix invasion in the MCF-7 breast carcinoma cell line transfected with individual or pooled siRNAs targeting HOTAIR. FIG. 2C shows the number of lung metastasis of vector or HOTAIR LincRNA expressing cells 9 weeks after injection of cells in the tail vein of nude mice. FIG. 2D shows representative photomicrographs of H&E stained sections of lung tissue from vector or HOTAIR LincRNA injected mice. Arrow highlights a metastatic focus.

FIGS. 3A-3E show data showing that HOTAIR LincRNA promotes selective, genome-wide, re-targeting of PRC2 and H3K27me3. FIG. 3A shows a heat map representing genes with a significant relative change in chromatin occupancy of EZH2, SUZ12, and H3K27 following HOTAIR expression. MDA-MB-231 vector or HOTAIR LincRNA cells were subjected to chromatin immunoprecipitation (ChIP) using anti-EZH2, H3K27me3, and SUZ12 antibodies followed by interrogation on a genome-wide promoter array. Values are depicted as relative ratio of HOTAIR LincRNA over vector cells and represented as a light-dark scale. FIG. 3B shows the top 5 enriched Gene Ontologies of the 854 genes with a gain of PRC2 occupancy and H3K27me3 following enforced expression of HOTAIR LincRNA. FIG. 3C average SUZ12 occupancy of >800 PRC-2 target genes in HOTAIR LincRNA or vector expressing cells across the length of gene promoter and gene body. All target genes are aligned by their transcriptional start sites (TSS). FIG. 3D show SUZ12 occupancy measured by ChIP-qPCR in vector or HOTAIR expression for the indicated gene promoters. Mean+s.d. are shown. FIG. 3E show module map20 of the 854 genes with a gain in PRC2 occupancy following HOTAIR LincRNA overexpression. (Left panel) Heat map of genes (column) showing a gain in PRC2 occupancy following HOTAIR expression in breast carcinoma cells [see panel (FIG. 3A)] compared with PRC-2 occupancy patterns from the indicated cell or tissue type (rows). Binary scale is dark gray (match) or light gray (no match). (Right panel) Quantification of significance of pattern matching between gene sets.

FIGS. 4A-4F show that HOTAIR LincRNA-induced matrix invasion and global gene expression changes requires PRC2. FIG. 4A show an immunoblot of SUZ12 and EZH2 protein levels following transduction of MDA-MB-231 vector or HOTAIR cells with retrovirus expressing a shRNA targeting either GFP, EZH2, or SUZ12. FIG. 4B show that matrix invasion in vector or HOTAIR cells expressing the indicated shRNA. Mean+s.d. are shown. FIG. 4C show (left panel) Heat map of gene with significant induction (gray) or repression (dark gray) following HOTAIR LincRNA expression in the MDA-MB-231 cells (right panel) The relative expression of the same gene list in MDA-MB-231 HOTAIR LincRNA cells expressing shEZH2 or shSUZ12 (expressed as a ratio to HOTAIR cells expressing shGFP). FIG. 4D show that qRT-PCR of a representative panel of genes in MDA-MB-23 1 vector or HOTAIR LincRNA cells also expressing the indicated shRNA. FIG. 4E show that matrix invasion in the immortalized H16N2 breast epithelial line expressing vector or EZH2 as well as EZH2-expressing cells transfected with siRNAs targeting GFP or HOTAIR LincRNA. FIG. 4F shows a working model of the role of HOTAIR LincRNA in breast cancer progression. Selection for increased HOTAIR LincRNA expression in a subset of breast primary tumors leads to a genome-wide retargeting of the PRC2 and H3K27me3 patterns, resulting in gene expression changes that promote tumor metastasis.

FIG. 5 shows higher resolution of subsets (i) and (ii) from the heat map depicted in FIG. 1A identifying transcripts that show (i) higher expression in normal compared to cancer samples and (ii) higher expression in primary compared to metastatic samples.

FIG. 6 is a heat map (supervised hierarchical clustering) representing the relative expression values of a filtered subset of HOX coding genes and lincRNAs as determined by qRT-PCR. RNA from 88 samples (5 normal breast organoid, 78 primary breast tumors from the NKI 295 Cohort, and 5 metastatic breast tumors) was assayed for the expression of 43 HOX lincRNAs and 39 HOX coding genes by qRT-PCR. Transcripts were filtered for significant differences in expression (SAM, 300 permutations, FDR<5%).

FIG. 7 gives relative levels of HOTAIR LincRNA by qRT-PCR of the indicated breast carcinoma cell line. Values are expressed relative to HOTAIR abundance in human adult foot fibroblast cells; error bars represent s.d. (n=3).

FIG. 8 shows levels of HOTAIR LincRNA following enforced expression in MCF-10A, SK-BR3, and MDA-MB-231 cells in relationship to HOTAIR LincRNA levels in the 132 primary breast tumors screened in FIG. 1D (measured on same scale in both left and right panel to allow direct comparison); error bars represents s.d. (n=3).

FIGS. 9A-9C show data from soft agar colony counts (in either 2% or 10% FBS) in HCC1954, SK-BR3 and MDA-MB-231 cells after transduction with vector or HOTAIR. FIG. 9A shows HCC1954 cells after transduction with vector or HOTAIR LincRNA. FIG. 9B shows SK-BR3 cells after transduction with vector or HOTAIR LincRNA. FIG. 9C show MDA-MB-231 cells after transduction with vector or HOTAIR. Assays were repeated in triplicate and mean±s.e.m. are shown. Statistical significance (highlighted by *) was determined by paired t-test (p values: HCC1954 2%=0.007, HCC1954 10%=0.003, SK-BR32%=0.003, SK-BR310%=0.001, MDA-MB-23 1 2%=0.011).

FIGS. 10A-10B show the relative levels of HOTAIR LincRNA after transfection with siRNA duplexes targeting HOTAIR LincRNA. FIG. 10A shows qRT-PCR results showing the relative levels of HOTAIR in the MCF-7 line after transfection with siRNA duplexes targeting HOTAIR. Matrix invasion of the same cells were shown in FIG. 2B. FIG. 10B shows the relative levels of HOTAIR LincRNA (by qRT-PCR) in the H16N2 cell line infected with retroviral vector or EZH2 before and after transfection with siRNA duplexes targeting HOTAIR LincRNA (either pooled or two individual duplexes). Matrix invasion of the same cells were shown in FIG. 4e. Error bars represent s.d. (n=3).

FIGS. 11A-11B show HOTAIR LincRNA expression on micromatastaic lesions. FIG. 11A shows in situ hybridization of HOTAIR using either HOTAIR antisense (AS) or sense (S) cRNA probes on a micrometastatic lesion in mouse lung following tail vein injection of MDA-MB-231 HOTAIR cells showing retention of HOTAIR LincRNA expression post-injection. FIG. 11B shows RT-PCR of HOTAIR and GAPDH from RNA isolated from micrometastatic lesions in mouse lung following tail vein injection of MDA-MB-231 HOTAIR or Vector cells.

FIG. 12 shows the Top 5 enriched Gene Ontologies of the 854 genes with a gain of PRC2 occupancy and H3K27me3 following enforced expression of HOTAIR LincRNA.

FIGS. 13A-13C shows occupancy of promoters of SUZ12, H3K27, and EZH2 genes by HOTAIR LincRNA. FIG. 13A shows occupancy of the SUZ12 promoter measured by ChIP-qPCR in vector or HOTAIR LincRNA cells. FIG. 13B shows occupancy of the H3K27 promoter measured by ChIP-qPCR in vector or HOTAIR LincRNA cells. FIG. 13C shows occupancy of the EZH2 promoter measured by ChIP-qPCR in vector or HOTAIR cells. Mean±s.d. are shown (n=3).

FIGS. 14A-14B show a gain of PRC-2 occupancy upon enforced HOTAIR LincRNA expression in the MDA-MB-231 cells. FIG. 14A shows a heat Map representing unsupervised hierarchical clustering of the relative expression values from 295 primary breast tumors (NKI 295 Cohort) of the 854 promoter set (from FIG. 3A) that show a gain of PRC-2 occupancy upon enforced HOTAIR LincRNA expression in the MDA-MB-23 1 cells. A subset of patients (dark bar—HOTAIR-PRC-2 targets DOWN) was identified that show a consistent down-regulation (relative silencing) of a subset of genes from this set. FIG. 14B show Kaplan-Meier curves showing overall survival in patients with the expression signature of HOTAIR PRC-2 targets UP (top line) or DOWN (bottom line) as delineated in the upper panel heat map.

FIG. 15 is a diagram of an embodiment of a system for performing a method for assessing the for HOTAIR expression in a biological sample obtained from a subject.

FIG. 16 is a diagram of an embodiment of a comparison module as described herein.

FIG. 17 is a diagram of an embodiment of an operating system and applications for a computing system as described herein.

### DETAILED DESCRIPTION OF THE INVENTION

The present invention generally relates to methods and composition to treat metastatic cancer comprising an inhibitor of the function of HOTAIR LincRNA and/or an inhibitor of HOTAIR LincRNA from the HOTAIR gene.

Alternative aspects of the present invention relate to methods for detecting a metastatic cancer in a subject, comprising measuring HOTAIR LincRNA expression in a biological sample from a subject, and comparing the measured level of HOTAIR LincRNA in the biological sample with a reference expression level for HOTAIR LincRNA and if there is an increased level of HOTAIR LincRNA in the biological sample obtained from the subject as compared to a reference level of HOTAIR LincRNA, the subject is identified as having a likelihood of increased metastatic cancer, and/or a decreased prognosis.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, a “subject” is any organism or animal to whom which treatment or prophylaxis treatment is desired. Such animals include mammals, preferably a human. The term “subject” also refers to any living organism from which a biological sample can be obtained. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “mammal” or “mammalian” are used interchangeably herein, are intended to encompass their normal meaning. While the invention is most desirably intended for efficacy in humans, it may also be employed in domestic mammals such as canines, felines, and equines, as well as in mammals of particular interest, e.g., zoo animals, farmstock, transgenic animals, rodents and the like.

As used herein, “gene silencing” or “gene silenced” in reference to an activity of a RNAi molecule, for example a siRNA or miRNA refers to a decrease in the mRNA level in a cell for a heterologous target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. As used herein, the “reduced” or “gene silencing” refers to lower, preferably significantly lower, more preferably the expression of the nucleotide sequence is not detectable.

The term “double-stranded RNA” molecule, “RNAi molecule”, or “dsRNA” molecule as used herein refers to a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. In some embodiments, the terms refer to a double-stranded RNA molecule capable, when expressed, is at least partially reducing the level of the mRNA of the heterologous target gene. In particular, the RNAi molecule is complementary to a synthetic RNAi target sequence located in a non-coding region of the heterologous target gene. As used herein, “RNA interference”, “RNAi”, and “dsRNAi” are used interchangeably herein refer to nucleic acid molecules capable of gene silencing.

As used herein, the term “RNAi” refers to any type of interfering RNA, including but are not limited to, siRNAi, shRNAi, stRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length).

As used herein “shRNA” or “small hairpin RNA” (also called stem loop) is a type of siRNA. In one embodiment, these shRNAs are composed of a short, e.g. about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches. In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

As used herein the term “hairpin RNA” refers to any self-annealing double stranded RNA molecule. In its simplest representation, a hairpin RNA consists of a double stranded stem made up by the annealing RNA strands, connected by a single stranded RNA loop, and is also referred to as a “pan-handle RNA”. However, the term “hairpin RNA” is also intended to encompass more complicated secondary RNA structures comprising self-annealing double stranded RNA sequences, but also internal bulges and loops. The specific secondary structure adapted will be determined by the free energy of the RNA molecule, and can be predicted for different situations using appropriate software such as FOLDRNA (Zuker and Stiegler (1981) Nucleic Acids Res 9(1):133-48; Zuker, M. (1989) Methods Enzymol. 180, 262-288).

The term “agent” refers to any entity which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “a reduction” of the level of a gene, included a decrease in the level of a protein or mRNA means in the cell or organism. As used herein, “at least a partial reduction” of the level of an agent (such as a RNA, mRNA, rRNA, tRNA expressed by the target gene and/or of the protein product encoded by it) means that the level is reduced at least 25%, preferably at least 50%, relative to a cell or organism lacking the RNAi agent as disclosed herein. As used herein, “a substantial reduction” of the level of an agent such as a protein or mRNA means that the level is reduced relative to a cell or organism lacking a chimeric RNA molecule of the invention capable of reducing the agent, where the reduction of the level of the agent is at least 75%, preferably at least 85%. The reduction can be determined by methods with which the skilled worker is familiar. Thus, the reduction of the transgene protein can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) to detect transgene protein or mRNA. Depending on the type of the reduced transgene, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

In its broadest sense, the term “substantially identical”, when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of 10-22 nucleotides, such as at least 10, 11, 12, 13, 14, 15, 16, 17, 18, 19, 20, 21, 22 or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially identical” to a reference nucleotide sequence hybridizes to the exact complementary sequence of the reference nucleotide sequence (i.e. its corresponding strand in a double-stranded molecule) under low stringency conditions, preferably medium stringency conditions, most preferably high stringency conditions (as defined above). Homologues of a specific nucleotide sequence include nucleotide sequences that encode an amino acid sequence that is at least 24% identical, at least 35% identical, at least 50% identical, at least 65% identical to the reference amino acid sequence, as measured using the parameters described above, wherein the amino acid sequence encoded by the homolog has the same biological activity as the protein encoded by the specific nucleotide. The term “substantially non-identical” refers to a nucleotide sequence that does not hybridize to the nucleic acid sequence under stringent conditions. The term “substantially identical”, when used herein with respect to a polypeptide, means a protein corresponding to a reference polypeptide, wherein the polypeptide has substantially the same structure and function as the reference protein, e.g. where only changes in amino acids sequence not affecting the polypeptide function occur. When used for a polypeptide or an amino acid sequence, the percentage of identity between the substantially similar and the reference polypeptide or amino acid sequence is at least 24%, at least 30%, at least 45%, at least 60%, at least 75%, at least 90%, at least 95%, at least 99%, using default GAP analysis parameters as described above. Homologues are amino acid sequences that are at least 24% identical, more preferably at least 35% identical, yet more preferably at least 50% identical, yet more preferably at least 65% identical to the reference polypeptide or amino acid sequence, as measured using the parameters described above, wherein the amino acid sequence encoded by the homolog has the same biological activity as the reference polypeptide.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

The terms “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. The term “tumor” or “tumor cell” are used interchangeably herein, refers to the tissue mass or tissue type of cell that is undergoing abnormal proliferation.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “treatment” refers to any treatment of a pathologic condition in a subject, particularly a human subject, and includes one or more of the following: (a) preventing a pathological condition from occurring in a subject which may be predisposition to the condition but has not yet been diagnosed with the condition and, accordingly, the treatment constitutes prophylactic treatment for the disease or condition; (b) inhibiting the pathological condition, i.e. arresting its development, (c) relieving the pathological condition, i.e. causing a regression of the pathological condition; or (d) relieving the conditions mediated by the pathological condition.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “optional” or “optionally” means that the subsequent described event, circumstance or substituent may or may not occur, and that the description includes instances where the event or circumstance occurs and instances where it does not.

Other than in the operating examples, or where otherwise indicated, all numbers expressing quantities of ingredients or reaction conditions used herein should be understood as modified in all instances by the term “about.” The term “about” when used in connection with percentages can mean±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

In this specification and the appended claims, the singular forms “a,” “an,” and “the” include plural references unless the context clearly dictates otherwise, and therefore “a” and “an” are used herein to refer to one or to more than one (i.e., at least one) of the grammatical object of the article. By way of example, “an element” means one element or more than one element, and reference to a composition for delivering “an agent” includes reference to one or more agents.

Compositions or methods “comprising” one or more recited elements may include other elements not specifically recited. For example, a composition that comprises an inhibitor of HOTAIR encompasses both an inhibitor of HOTAIR but may also include other agents or other components. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

Other than in the operating examples, or where otherwise indicated, all numbers expressing quantities of ingredients or reaction conditions used herein should be understood as modified in all instances by the term “about.” The term “about” is used herein to mean approximately, roughly, around, or in the region of. When the term “about” is used in conjunction with a numerical range, it modifies that range by extending the boundaries above and below the numerical values set forth. The term “about” when used in connection with percentages will mean±1%.

The present invention is not limited to the particular methodology, protocols, and reagents, etc., described herein, as such may vary. The terminology used herein is for the purpose of describing particular embodiments only, and is not intended to limit the scope of the present invention, which is defined solely by the claims. Unless defined otherwise, all technical and scientific terms used herein have the same meaning as those commonly understood to one of ordinary skill in the art to which this invention pertains.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These references are provided solely for their disclosure, and nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Linc RNA

Without wishing to be bound by theory, large intergenic noncoding RNAs (lincRNAs, also called long ncRNAs) are generally considered as non-protein coding transcripts longer than 200 nucleotides. This (somewhat arbitrarily) size limit is due to practical considerations including the separation of RNAs in common experimental protocols. Additionally, the size limit distinguishes lincRNAs from small regulatory RNAs, such as microRNAs (miRNAs), short interfering RNAs (siRNAs), Piwi-interacting RNAs (piRNAs), small nucleolar RNAs (snoRNAs), etc. LincRNAs are pervasively transcribed in the genome (Amaral et al., 319 Sci. 1787-89 (2008); Carninci et al., 309 Sci. 1559-63 (2005); Guttman et al., 458 Nat. 223-37 (2009)), yet their potential involvement in human disease is not well understood (Calin et al., 12 Canc. Cell 215-29 (2007); Yu et al., 451 Nat. 202-06 (2008)). Recent studies of dosage compensation, imprinting, and homeotic gene expression suggest that individual lincRNAs can function as the interface between DNA and specific chromatin remodeling activities. Ponting et al., 136 Cell 629-41 (2009); Rinn et al., 129 Cell 1311-23 (2007); Khalil et al., 106 PNAS 11675-80 (2009).

### HOTAIR LincRNA

The present inventors have discovered, as described herein, that lincRNAs in the HOX loci become systematically dysregulated during breast cancer progression. The inventors demonstrated that a particular lincRNA, termed HOX antisense intergenic RNA (HOTAIR LincRNA), is progressively increased in expression in primary breast tumors and metastases, and HOTAIR expression level in primary tumors is predictive of eventual metastasis and death. Enforced expression of HOTAIR LincRNA in epithelial cancer cells induced genome-wide re-targeting of Polycomb Repressive Complex 2 (PRC2) to an occupancy pattern more resembling embryonic fibroblasts, leading to altered histone H3 lysine 27 methylation, gene expression, and increased cancer invasiveness and metastasis in a manner dependent on PRC2. Conversely, the inventors demonstrated that loss of HOTAIR LincRNA, e.g., inhibition or downregulation of HOTAIR LincRNA can inhibit cancer invasiveness, particularly in cells that possess excessive PRC2 activity. Accordingly, the inventors have discovered that inhibition of HOTAIR LincRNA can be used to inhibit cancer invasiveness and cancer metastasis. These findings demonstrate that lincRNAs play active roles in modulating the cancer epigenome and may be important targets for cancer diagnosis and therapy.

More specifically, in mammals, strong epigenetic mechanisms are thought to underlie the embryonic expression profiles of the HOX genes that persist throughout human development (Mazo et al., 120 J. Cell Sci. 2755-61 (2007); Rinn et al., 2007). The human HOX genes are associated with hundreds of ncRNAs that are sequentially expressed along both the spatial and temporal axes of human development and define chromatin domains of differential histone methylation and RNA polymerase accessibility. Rinn et al., 2007. HOTAIR LincRNA originates from the HOXC locus and represses transcription across 40 kb of the HOXD locus by altering chromatin trimethylation state. HOTAIR LincRNA is thought to achieve this by directing the action of Polycomb chromatin remodeling complexes, specifically the PRC2 complex, in trans, thus governing the cell's epigenetic state and subsequent gene expression. Although lincRNAs were thought previously to function by regulating chromatin states in cis (Ponting et al., 2009), the discovery of HOTAIR led to the recognition that lincRNAs can also regulate chromatin state on genes at a distance. Rinn et al., 2007. Components of PRC2 contain RNA binding domains that may potentially bind HOTAIR LincRNA and probably other similar lincRNAs. Denisenko et al., 18 Mol. Cell. Biol. 5634-42 (1998); Katayama et al., 309 Sci. 1564-66 (2005).

The full scope of trans regulation by a lincRNA and its possible relevance to human disease remained unknown before the present invention Likewise, the potential functions of many other lincRNAs in the human HOX loci were previously undefined. Perturbation of HOX coding and microRNA genes are frequently observed in breast and other cancers, and contributes to breast epithelial transformation (Rama et al., 405 Nat. 974-78 (2000), and breast cancer metastasis (Ma et al., 449 Nat. 682-88 (2007)). PRC2, comprised of core subunits EZH2, SUZ12 and EED, is a histone H3 lysine 27 methylase that is involved in developmental gene silencing, cellular self-renewal, and cancer progression. Sparmann & van Lohuizen, 6 Nat. Rev. Canc. 846-56 (2006). Notably, PRC2 subunit EZH2 is amplified or overexpressed in several human cancers, including breast cancer, and promotes breast cancer invasiveness. Kleer et al., 100 PNAS 11606-11 (2003). The inventors assessed if altered expression of one or more HOX lincRNAs is involved in human cancer, thereby promoting genomic relocalization of Polycomb complex and H3K27 trimethylation.

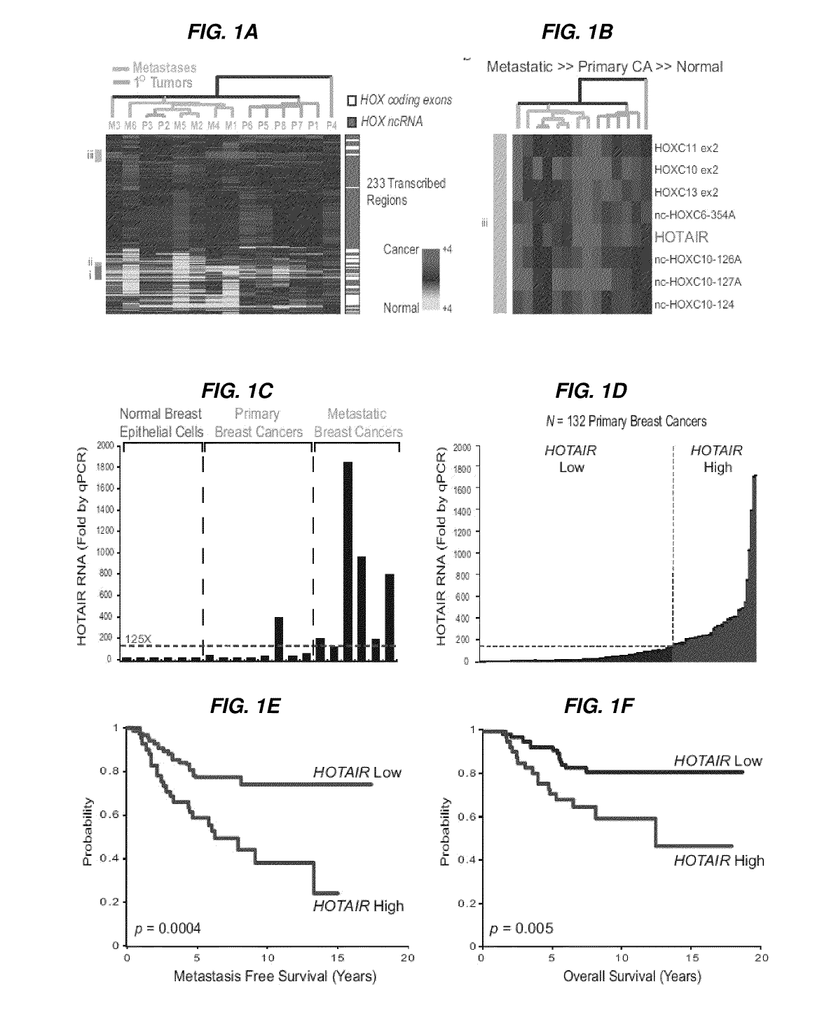

To determine whether HOX lincRNAs is dysregulated during cancer progression, the inventors hybridized RNA derived from normal human breast epithelia, primary breast carcinomas, and distant metastases to ultra-dense HOX tiling arrays (Rinn et al., 2007) (FIG. 1A, 1B). The inventors discovered that 233 transcribed regions in the HOX loci, comprising 170 ncRNAs and 63 HOX exons, were differentially expressed in these samples (FIG. 1a). Unsupervised hierarchical clustering of the expression data showed systematic variation in the expression of HOX lincRNAs among normal breast epithelia, primary tumor, and metastases. HOXA5, a known breast tumor suppressor (Rama et al., 2000), along with dozens of HOX lincRNAs, are expressed in normal breast but decreased in expression in all cancer samples (FIG. 5a). These lincRNAs are candidate tumor suppressor genes. A set of HOX lincRNAs and mRNAs, including the known oncogene HOXB7 (Wu et al., 66 Canc. Res. 9527-34 (2006)), are increased in expression in primary tumors but not in metastases (FIG. 5a). A distinct set of HOX lincRNAs show moderately increased expression in primary tumors, and are further increased in expression in metastases (FIG. 1b). Notably, one such metastasis-associated lincRNA is HOTAIR (FIG. 1b).

Quantitative PCR demonstrated that HOTAIR LincRNA is overexpressed from hundreds to nearly two thousand-fold in breast cancer metastases, and HOTAIR LincRNA level is also increased but heterogeneous among primary tumors (FIG. 1C). In order to test whether the level of HOTAIR LincRNA in primary breast tumors can predict metastasis, the inventors measured HOTAIR LincRNA level in an independent panel of 132 primary breast tumors (stage I and II) with extensive clinical follow-up. van de Vijver, 347 New Engl. J. Med. 1999-09 (2008). Indeed, nearly one third of primary breast tumors overexpress HOTAIR LincRNA by over 125-fold over normal breast epithelia, the minimum level of HOTAIR LincRNA overexpression observed in bona fide metastases (FIG. 1D), and high HOTAIR LincRNA level is a significant predictor of subsequent metastasis and death (p=0.0004 and p=0.005 for metastasis and death, respectively, FIGS. 1E, 1F). Multivariate analysis showed that prognostic stratification of metastasis and death by HOTAIR LincRNA is independent of known clinical risk factors such as tumor size, stage, and hormone receptor status (Table 1). Patients with primary tumors that exhibit high levels of HOTAIR LincRNA are 3.5-fold more likely to experience subsequent metastasis and 3-fold more likely to die over time. Thus, lincRNAs can exhibit profound transcriptional dysregulation in breast cancer, and the expression level of even a single lincRNA, such as HOTAIR, is strongly associated with metastatic potential.

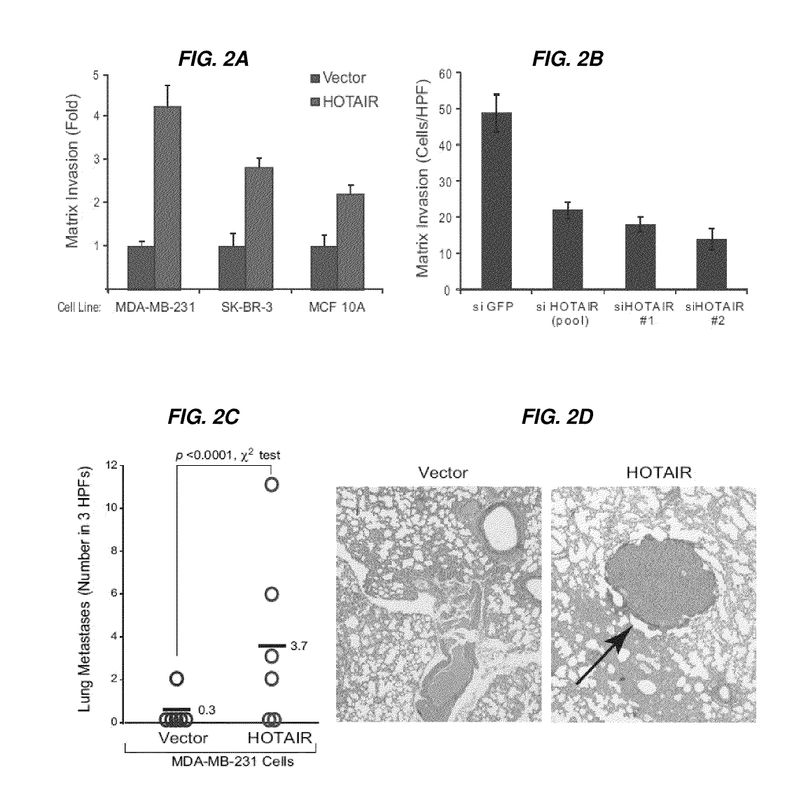

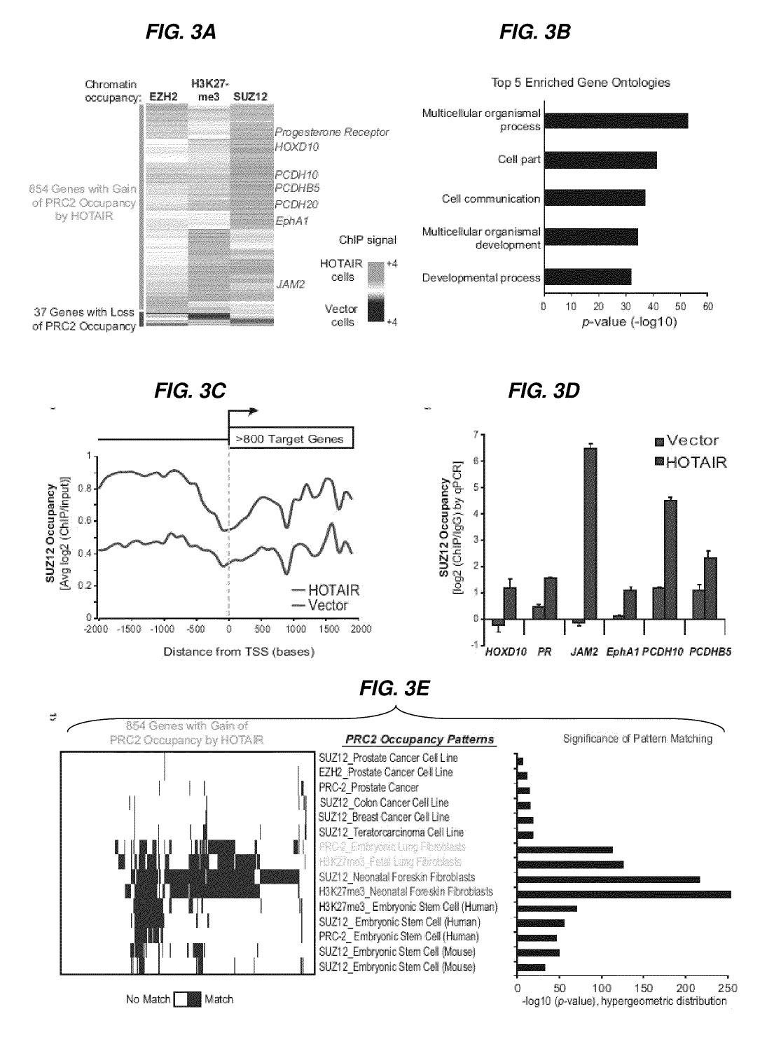

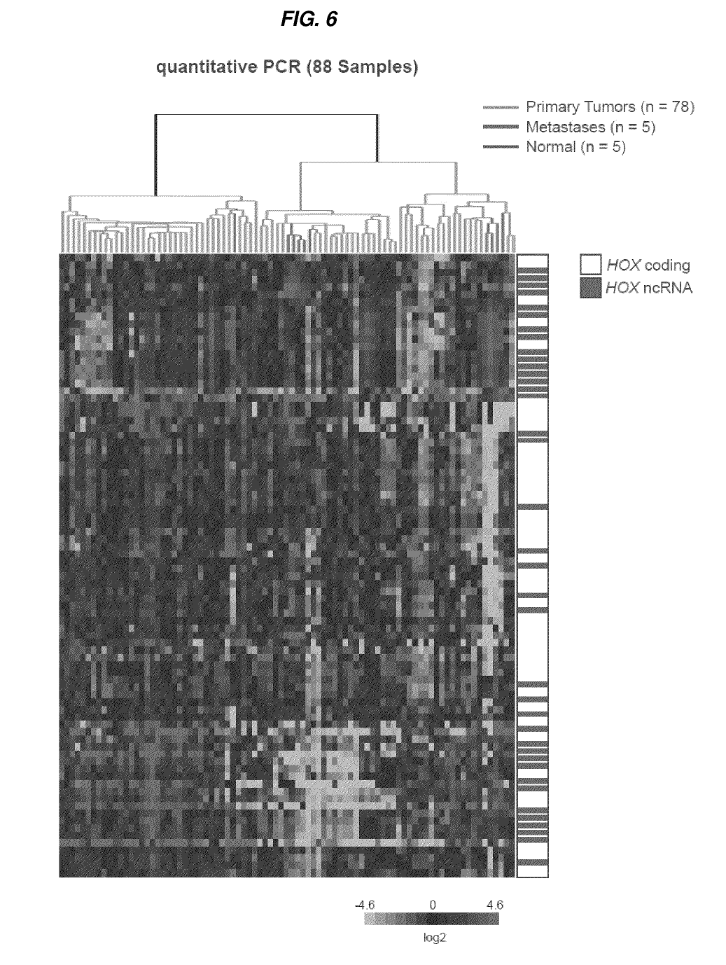

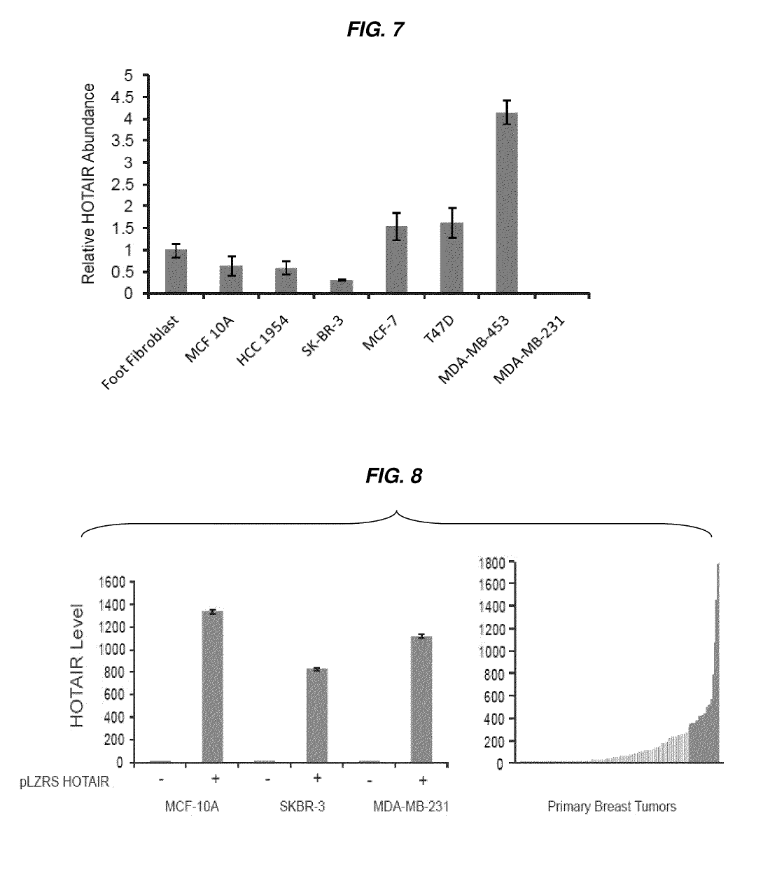

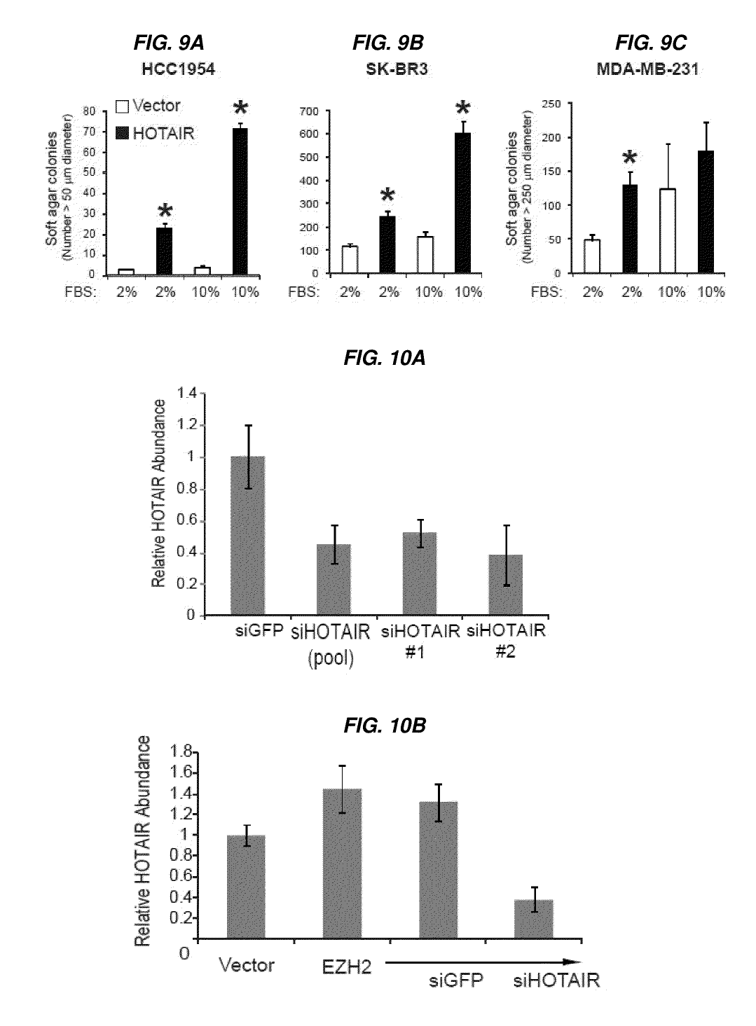

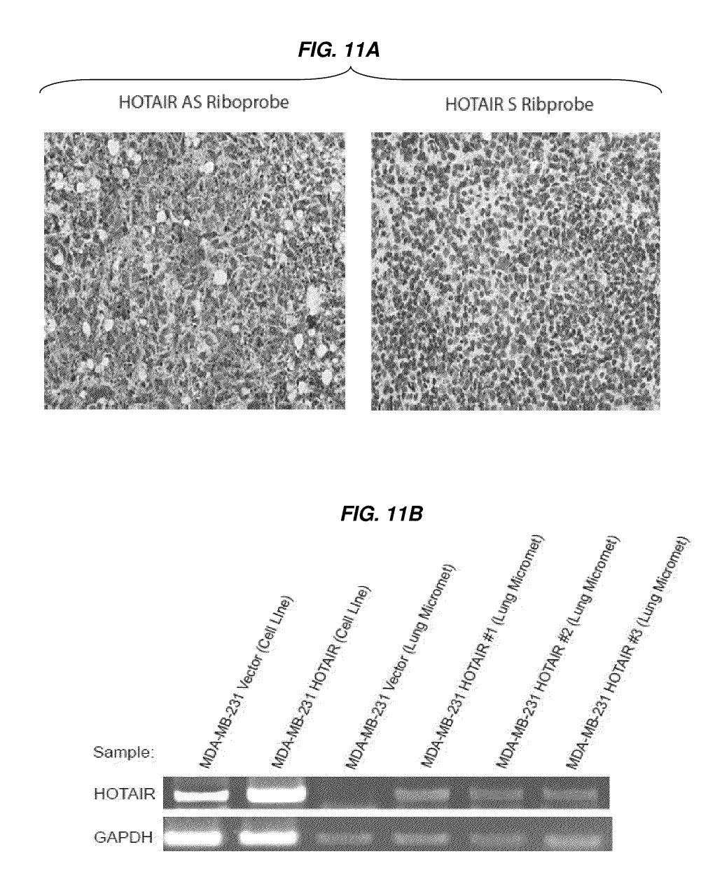

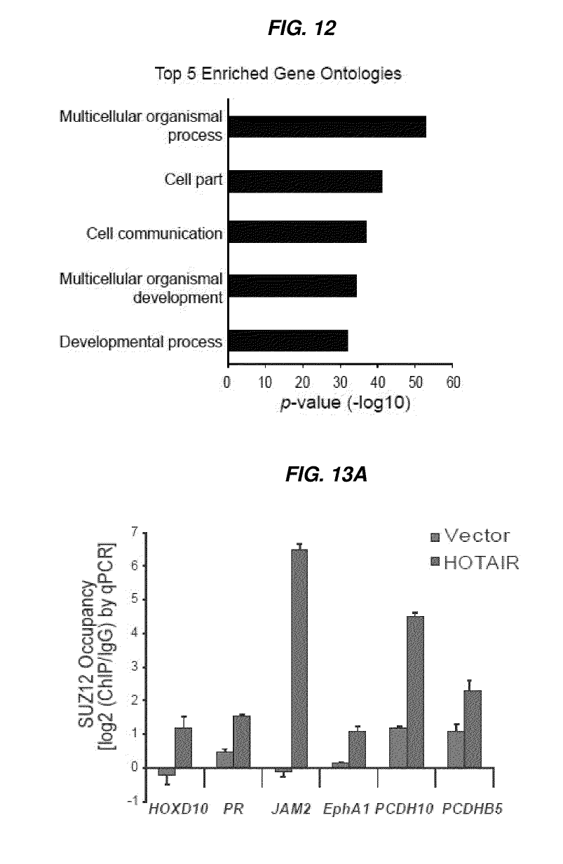

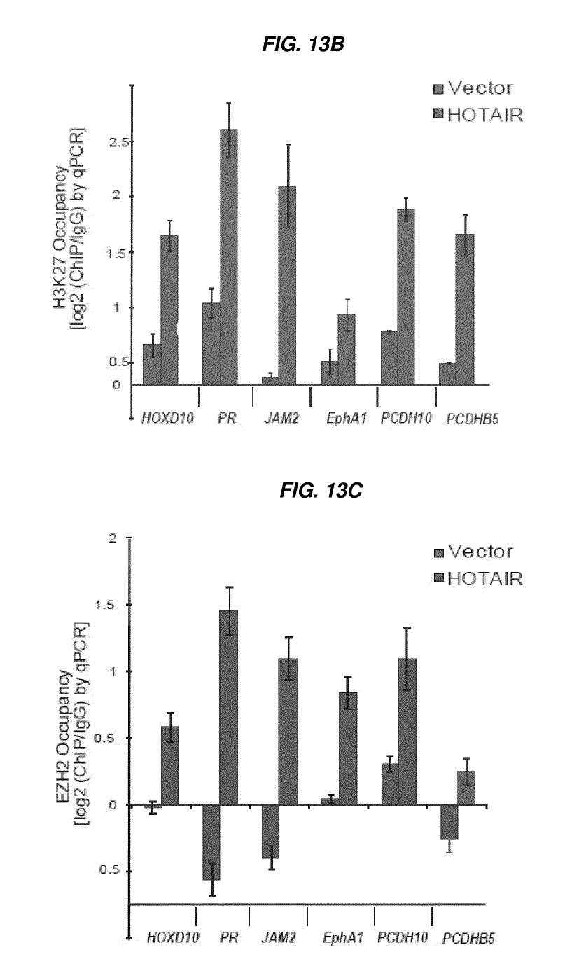

To probe the function of HOTAIR LincRNA in metastasis, the inventors examined the effects of manipulating HOTAIR LincRNA level in several breast cancer cell lines. HOTAIR LincRNA levels were determined in a panel of breast cancer cell lines (FIG. 6). Retroviral transduction in several lines allowed stable overexpression of HOTAIR LincRNA to several hundred fold over vector-transduced cells, which are comparable to levels observed in patients (FIG. 7). Stable enforced expression of HOTAIR LincRNA did not change cell proliferation in vitro or subcutaneous tumor xenograft growth in vivo (FIGS. 8a, 8b). Importantly, enforced expression of HOTAIR in three different breast cancer cell lines, representing both transformed and immortalized phenotypes, significantly increased cancer cell invasion through Matrigel, a basement-membrane like extracellular matrix, (FIG. 2A). Conversely, depletion of HOTAIR LincRNA by small inferring RNAs (siRNAs) in MCF7, a cell line that expresses high HOTAIR LincRNA, substantially decreased its matrix invasiveness (FIG. 2B). Two independent siRNAs targeting HOTAIR LincRNA each inhibited cell invasiveness and decreased HOTAIR LincRNA level (FIG. 2B, FIG. 9), suggesting that the requirement of HOTAIR LincRNA for cell invasion is unlikely to be an off-target effect. Next, to address whether HOTAIR LincRNA may potentiate metastatic potential in vivo, the inventors compared the efficiency of vector-transduced or HOTAIR LincRNA-transduced MDA-MB-231 breast cancer cells to metastasize to the lung after tail vein injection. Nine weeks after injection, microscopic analyses showed that whereas vector-transduced cells had produced few metastases, HOTAIR-expressing cells generated a robust number of metastases (p<0.0001, χ2 test, FIG. 3c). Together, these results suggest that HOTAIR LincRNA overexpression can increase cancer cell invasiveness and promote metastases in vivo.

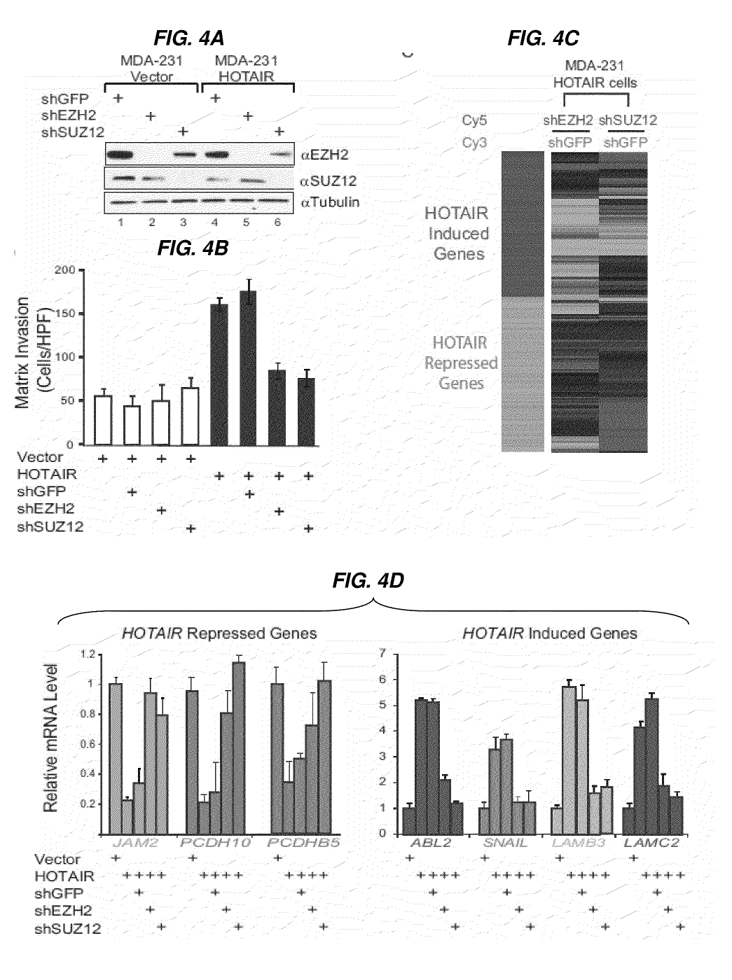

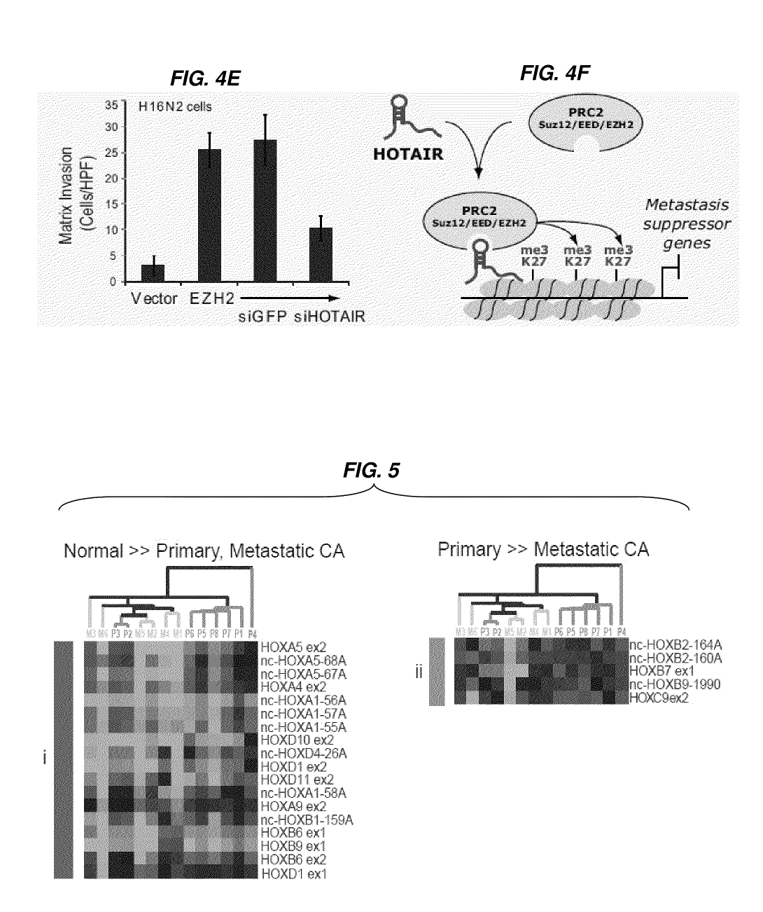

A key question is how a lincRNA, such as HOTAIR, can drive cancer metastasis. Because HOTAIR LincRNA physically interacts with PRC2, a histone modification complex implicated in cancer progression (Sparmann & van Lohuizen, 2006), and is required to target PRC2 to the HOXD locus (Rinn et al., 2007), the inventors tested if HOTAIR LincRNA overexpression led to retargeting of PRC2 to many regions of the genome. The inventors mapped PRC2 occupancy genome-wide by chromatin immunoprecipitation followed by hybridization to tiling microarrays interrogating all human promoters (ChIP-chip analysis, FIG. 3). Compared to vector expressing cells, MDA-MB-231 cells over-expressing HOTAIR LincRNA demonstrated increased occupancy of PRC2 subunits Suz12, EZH2, and increased H3K27me3 on 854 genes, while concomitantly losing PRC2 occupancy and H3K27me3 on 37 genes (FIG. 3A). The majority of PRC2 occupancy sites on promoters, genome-wide, showed little change, and HOTAIR LincRNA overexpression did not change the levels of PRC2 subunits (FIG. 4A, lane 1 vs. lane 4). Thus, the predominant effect of HOTAIR LincRNA overexpression is not to change the expression levels of the PRC2 subunits, but to cause the selective re-targeting of PRC2 and H3K27me3 within the genome. A number of the genes with HOTAIR-induced PRC2 occupancy are implicated in inhibiting breast cancer progression, including transcription factors HOXD1010 and PRG1, encoding progesterone receptor (a classic favorable prognostic factor); cell adhesion molecules of the protocadherin (PCDH) gene family (Novak et al., 68 Canc. Res. 8616-25 (2008)), and JAM2 (Naik et al., 68 Canc. Res. 2194-203 (2008)), and EPHA1 (Fox & Kandpal, 318 Biochem. Biophys. Res. Commn. 882-92 (2004); Herath et al., 100 Br. J. Canc. 1095-102 (2009)), encoding an ephrin receptor involved in tumor angiogenesis. Among the 854 genes with HOTAIR-induced PRC2 occupancy, Gene Ontology analysis (Ashburner et al., 25 Nat. Genet. 25-29 (2000)) suggested a majority of the genes are involved in pathways related to cell-cell signaling and development, consistent with the known critical role of HOX transcripts in body patterning (FIG. 3b). HOTAIR-induced PRC2 occupancy tended to spread over promoters, and to a lesser extent, gene bodies (FIG. 3C). ChIP followed by quantitative PCR confirmed that HOTAIR LincRNA substantially increased PRC2 occupancy of all target genes examined (FIG. 3D).

To gain further insight into why HOTAIR LincRNA induces PRC2 occupancy of a select set of genes, the inventors compared the 854 genes with HOTAIR-induced PRC2 occupancy in MDA-MB-231 cells with a compendium of published PRC2 occupancy profiles in diverse cell types (FIG. 3E). PRC2 occupancy patterns from different cancer, fibroblastic, and embryonic stem cell lines were annotated from existing databases (see Table 4 for references). Using a pattern matching algorithm (Segal et al., 36 Nat. Genet. 1090-98 (2004)), the inventors discovered that the HOTAIR-induced PRC2 occupancy pattern in breast cancer cells most resembled the endogenous PRC2 occupancy pattern in embryonic and neonatal fibroblasts, especially fibroblasts derived from posterior and distal anatomic sites (such as the foreskin), where endogenous HOTAIR LincRNA is expressed (Rinn et al., 2007) (p<10-50 for each comparison, FDR<<0.05, FIG. 3E). These results suggest that elevated HOTAIR expression in breast cancer cells appears to reprogram the Polycomb binding profile of a breast epithelial cell to that of an embryonic fibroblast.

Finally, the inventors assessed whether the ability of HOTAIR LincRNA to induce breast cancer invasiveness required an intact PRC2 complex. The inventors transduced vector- or HOTAIR-expressing MDA-MB-231 cells with short hairpin RNAs (shRNAs) targeting PRC2 subunits EZH2 or SUZ12. Immunoblot analyses confirmed efficient depletion of the targeted proteins (FIG. 4a). Depletion of either SUZ12 or EZH2 had little impact on the matrix invasiveness of vector-expressing MDA-MB-231 cells, but substantially reversed the ability of HOTAIR LincRNA to promote matrix invasion (FIG. 4B). This result demonstrates that PRC2 is specifically required for HOTAIR to promote cellular invasiveness. Global gene expression analysis revealed hundreds of genes that were induced or repressed as a consequence of HOTAIR LincRNA overexpression (FIG. 4C, left panel); interestingly, roughly the same number of genes was induced upon HOTAIR LincRNA expression, likely to due to secondary effects as they were not targets of HOTAIR-induced PRC2 occupancy. Importantly, concomitant depletion of PRC2 in large part reversed the global gene expression pattern to that of cells not overexpressing HOTAIR LincRNA (FIG. 4C, right panel). Quantitative RT-PCR confirmed that HOTAIR-induced PRC2 target genes, such as JAM2, PCDH10, PCDHB5, were transcriptionally repressed upon HOTAIR expression and de-repressed upon concomitant PRC2 depletion (FIG. 4D). HOTAIR-induced genes (via indirect mechanisms) were also reversed upon PRC2 depletion (FIG. 4D). Of note, many of the genes induced by HOTAIR LincRNA are known positive regulators of cancer metastasis, including ABL221, SNAIL22, and laminins (Marinkovich, 7 Nat. Rev. Canc. 370-80 (2007)). Conversely, overexpression of EZH2 in H16N2 breast cells is known to promote matrix invasion (Kleer et al., 2003), but concomitant depletion of endogenous HOTAIR LincRNA in large measure inhibited the ability of EZH2 to induce matrix invasion (FIG. 4E and FIG. 10). Together, these results demonstrate a functional inter-dependency between HOTAIR and PRC2 in promoting cancer invasiveness.

### Inhibition of HOTAIR for the Treatment of Cancers

Thus, one aspect of the present invention relates to methods and compositions for inhibiting HOTAIR expression and signaling (e.g., HOTAIR-PRC2 signaling) in human cells. The method includes: (optionally) identifying a cell in which a reduction of the activity or level of HOTAIR is desired; and contacting said cell or cell population with an amount of a HOTAIR antagonist(s) sufficient to inhibit the activity or level of HOTAIR in the cell. The contacting step may be carried out ex vivo, in vitro, or in vivo. For example, the contacting step may be performed using human cells, or performed in a human patient.

The term “HOTAIR” refers to the hox transcript antisense RNA (non-protein coding) 1 2, and is also known by aliases: HOXAS1 2, NCRNA000721 2 and FLJ417472. HOTAIR can be identified as Entrez Gene ID: 100124700 and NCBI ref Sequence: NR_{—}003716.

In some embodiments of all aspects of the invention, an inhibitor of HOTAIR can be a nucleic acid inhibitor, such as an oligonucleotide, antisense, a RNAi molecule, such as but not limited to siRNA, miRNA, shRNA and the like, which gene silences a cancer target gene.

The methods and compositions, e.g., antagonists of HOTAIR, described herein are useful in treating cancer, such as breast cancer (e.g., ameliorating, delaying or preventing the onset of, or preventing recurrence or relapse of), or preventing progression of such cancer, by inhibiting metastasis. The antagonist may be a RNAi of HOTAIR lincRNA, such as siRNA, miRNA, shRNA, stRNA, snRNA, or antisense oligonucleotides. In humans, RNAi can be delivered systemically, for example, via targeted nanoparticles, see Davis et al., 464 Nature 1067-70 (2010). RNAi therapy has also been delivered successfully to humans by other routes, see, e.g., DeVincenzo et al., 107 PNAS 8800-05 (2010); Tiemann & Rossi, 1 EMBO Mol. Med. 142-51 (2009).

In some embodiments, an inhibitor of HOTAIR can inhibit is function by inhibiting its binding with polycomb (PRC2), where PRC2 is comprised of EZH2, SUZ12, EED. In some embodiments, an inhibitor of HOTAIR can inhibit is function by inhibiting its binding with LSD1, CoRest/Rest. In alternative embodiments, an inhibitor of HOTAIR can inhibit is binding to one or more target genes, e.g., HOXD genes.

The design of RNAi targets is known in the art. Generally, targeted regions are identified on a DNA sequence of a targeted gene about 50 to 100 nucleotides downstream of a promoter. A sequence motif is identified having the characteristic motif AA(N₁₉)TT, or NA(N₂₁), or NAR(N₁₇)YNN, where N is any nucleotide, R is a purine (A, G) and Y is a pyrimidine (C, U). Typically, the design avoids sequences with >50% G+C content, stretches of four or more nucleotide repeats, and sequences that share a certain degree of homology with other related or unrelated genes. RNAi design software is freely available, for example, SIDESIGN® CENTER (Dharmacon RNAi Technologies, Thermo Scientific, Worcester, Mass.) and Gene-Specific siRNA Selector (Wistar Bioinformatics, PA).

Thus, in some embodiments, the antagonist of HOTAIR is an oligonucleotide. In the context of this invention, the term “oligonucleotide” refers to a polymer or oligomer of nucleotide or nucleoside monomers consisting of naturally occurring bases, sugars and intersugar linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases. An oligonucleotide can be single-stranded or double-stranded. A single-stranded oligonucleotide can have double-stranded regions and a double-stranded oligonucleotide can have single-stranded regions. Single-stranded and double-stranded oligonucleotides that are effective in inducing RNA interference are referred to as siRNA, RNAi agent, iRNA agent, or RNAi inhibitor herein. These RNA interference inducing oligonucleotides associate with a cytoplasmic multi-protein complex known as RNAi-induced silencing complex (RISC). In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g. a target mRNA. Exemplary oligonucleotides include single-stranded and double-stranded siRNAs and other RNA interference reagents (RNAi agents or iRNA agents), shRNA (short hairpin RNAs), antisense oligonucleotides, ribozymes, microRNAs (miRNAs), microRNA mimics, triplex-forming oligonucleotides, and decoy oligonucleotides.

Oligonucleotides of the present invention can be of various lengths. In particular embodiments, oligonucleotides can range from about 10 to 100 nucleotides in length, inclusive. The oligonucleotides of the invention can comprise any oligonucleotide modification described herein and below. In certain instances, it can be desirable to modify one or both strands of a double-stranded oligonucleotide. In other instances, multiple different modifications can be included on each of the strands.

Double-stranded oligonucleotides comprising a duplex structure of between 20 and 23 base pairs, specifically 21 base pairs, have been hailed as particularly effective in inducing RNA interference (Elbashir et al., 20 EMBO 6877-88 (2001)). Others have found that shorter or longer double-stranded oligonucleotides can be effective as well. The double-stranded oligonucleotides comprise two oligonucleotide strands that are sufficiently complementary to hybridize to form a duplex structure. Generally, the duplex structure is between 15 and 30 base pairs in length. Alternatively, shorter double-stranded oligonucleotides of between 10 and 15 base pairs in length are used. In some embodiments, the double-stranded oligonucleotide is at least 21 nucleotides long. In some embodiments, the double-stranded oligonucleotide comprises a sense strand and an antisense strand, wherein the antisense RNA strand has a region of complementarity which is complementary to at least a part of a target sequence, and the duplex region is 14 to 30 nucleotides in length.

One or both ends of the double-stranded oligonucleotide can comprise a single-stranded overhang of 1 to 4 nucleotides, such as 1 or 2 nucleotides. As used herein, the term “overhang” refers to a double-stranded structure where at least one end of one strand is longer than the corresponding end of the other strand forming the double-stranded structure In some embodiments, the single-strand overhang sequence is 5′-dTdT-3′.

The phrase “antisense strand” as used herein, refers to an oligonucleotide that is substantially or 100% complementary to a target sequence of interest. The phrase “antisense strand” includes the antisense region of both oligonucleotides that are formed from two separate strands, as well as unimolecular oligonucleotides that are capable of forming hairpin or dumbbell type structures. The terms “antisense strand” and “guide strand” are used interchangeably herein.

The phrase “sense strand” refers to an oligonucleotide that has the same nucleoside sequence, in whole or in part, as a target sequence such as a messenger RNA or a sequence of DNA. The terms “sense strand” and “passenger strand” are used interchangeably herein.

By “target sequence” is meant any nucleic acid sequence whose expression or activity is to be modulated. The target nucleic acid can be DNA or RNA, such as HOTAIR or lincRNA.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII 123-33 (1987); Frier et al., 83 PNAS 9373-77 (1986); Turner et al., 109 J. Am. Chem. Soc. 3783-85 (1987)). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, inclusive). “Perfectly complementary” or 100% complementarity means that all the contiguous residues of a nucleic acid sequence will hydrogen bond with the same number of contiguous residues in a second nucleic acid sequence. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The double stranded siRNAs can also include double-stranded oligonucleotide wherein the two strands are linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23, inclusive. In some embodiments, the oligonucleotide linker is (dT)₄ or (U)₄.

Hairpin and dumbbell type RNAi agents will have a duplex region equal to or less than 200, 100, or 50 nucleotides in length. In some embodiments, ranges for the duplex region are 15 to 30, 17 to 23, 19 to 23, or 19 to 21 nucleotides pairs in length, inclusive. In some embodiments, the hairpin oligonucleotides can mimic the natural precursors of microRNAs. The hairpin RNAi agents can have a single strand overhang or terminal unpaired region, in some embodiments at the 3′, and in some embodiments on the antisense side of the hairpin. The two strands making up the hairpin structure can be arranged in any orientation. For example, the 3′-end of the antisense strand can be linked to the 5′-end of the sense strand, or the 5′-end of the antisense strand can be linked to the 3′-end of the sense strand. The hairpin oligonucleotides are also referred to as “shRNA” herein.

The single-stranded oligonucleotides can comprise nucleotide sequence that is substantially complementary to a “sense” nucleic acid encoding a gene expression product, e.g., complementary to the coding strand of a double-stranded DNA molecule or complementary to an RNA sequence, e.g., messenger RNA. The single-stranded oligonucleotides include antisense oligonucleotides and single-stranded RNAi agents. The region of complementarity is less than 30 nucleotides in length, and at least 15 nucleotides in length. Generally, the single stranded oligonucleotides are 10 to 25 nucleotides in length. Single strands having less than 100% complementarity to the target sequence are also embraced by the present invention.

An antisense single-stranded oligonucleotide can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition to, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule, but it can include a duplexed region, formed by intra-strand pairing, e.g., it can be, or include, a hairpin or pan-handle structure.

MicroRNAs (miRNAs or mirs) are a highly conserved class of small RNA molecules that are transcribed but are not translated into protein. Pre-microRNAs are processed into miRNAs. Processed microRNAs are single stranded ˜17 to 25 nucleotide RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. A number of miRNA sequences have been identified to date. See e.g., Griffiths-Jones et al., 34 NAR Database Issue D140-44 (2006); Griffiths-Jones 32 NAR Database Issue, D109-11 (2004).

miRNA mimics represent oligonucleotides that can be used to imitate the gene modulating activity of one or more miRNAs. Thus, the term “microRNA mimic” refers to synthetic non-coding RNAs (i.e., the miRNA is not obtained by purification from a source of the endogenous miRNA) that are capable of entering the RNAi pathway and regulating gene expression. miRNA mimics can be designed as mature molecules (e.g., single stranded) or mimic precursors (e.g., pri- or pre-miRNAs). In one design, miRNA mimics are double stranded molecules (e.g., with a duplex region of between about 16 to 31 nucleotides in length) and contain one or more sequences that have identity with the mature strand of a given miRNA. Double-stranded miRNA mimics have designs similar to as described above for double-stranded oligonucleotides.

The RNAi of the present invention may also be effected by ribozymes, which are oligonucleotides having specific catalytic domains that possess endonuclease activity. See Kim & Cech, 84 PNAS 8788-92 (1987); Forster & Symons, 49 Cell 211-20 (1987). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Methods of producing a ribozyme targeted to any target sequence are known in the art. WO 93/23569; WO 94/02595.

Decoy oligonucleotides may also be used to effect RNAi. Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to up-regulate genes that are suppressed by the binding of a transcription factor. Examples of the utilization of decoy oligonucleotides can be found in Mann et al., 106 J. Clin. Invest. 1071-75 (2000).

The terms “antimir” “microRNA inhibitor” or “miR inhibitor” are synonymous and refer to oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands.

MicroRNA inhibitors, including hairpin miRNA inhibitors, are described. See Vermeulen et al., 13 RNA 723-30 (2007); WO2007/095387; WO 2008/036825. A person of ordinary skill in the art can select a sequence from the database for a desired miRNA and design an inhibitor useful for the methods disclosed herein.

Alternatively, recent studies have shown that triplex forming oligonucleotides (TFO) can be designed which can recognize and bind to polypurine/polypyrimidine regions in double-stranded helical DNA in a sequence-specific manner. See Maher et al., 245 Sci. 725-30 (1989); Moser et al., 238 Sci. 645-30 (1987); Beal et al., 251 Sci. 1360-63 (1992); Conney et al., 241 Sci. 456-59 (1988); Hogan et al., EP 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences. See Seidman & Glazer, 1 J. Clin. Invest. 487-94 (2003). In general, the triplex-forming oligonucleotide has the sequence correspondence: oligo 3′-A G G T (SEQ ID NO: 2); duplex 5′-A G C T (SEQ ID NO: 3); duplex 3′-T C G A (SEQ ID NO: 38)

It has been shown that the A-AT and G-GC triplets have the greatest triple helical stability. TFOs designed according to the A-AT and G-GC rule do not form non-specific triplexes, indicating that the triplex formation is indeed sequence specific. Reither & Jeltsch, BMC Biochem. (Epub Sep. 12, 2002). Thus for any given sequence a triplex forming sequence can be devised. Triplex-forming oligonucleotides may be at least 15 or more nucleotides in length, up to 50 or 100 nucleotides, inclusive.

Without being bound by theory, formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFG1 and endogenous HPRT genes in mammalian cells (Vasquez et al., 27 Nucl. Acids Res. 1176-81 (1999); Puri, et al., 276 J Biol Chem. 28991-98 (2001)), and the sequence- and target specific downregulation of expression of the Ets2 transcription factor, important in prostate cancer etiology (Carbone et al, 31 Nucl. Acid Res. 833-43 (2003), and the pro-inflammatory ICAM-1 gene (Besch et al, 277 J. Biol. Chem. 32473-79 (2002)). In addition, it has been shown recently that sequence-specific TFOs can bind to dsRNA, inhibiting activity of dsRNA-dependent enzymes such as RNA-dependent kinases (Vuyisich & Beal, 28 Nucl. Acids Res 2000; 28; 2369-74 (2000). Additionally, TFOs designed according to the above-mentioned principles can induce directed mutagenesis capable of effecting DNA repair, thus providing both down-regulation and up-regulation of expression of endogenous genes. Seidman & Glazer, 112 J. Clin. Invest. 487-94 (2003). Detailed description of the design, synthesis and administration of effective TFOs is also known. U.S. Patent Pub. Nos. 2003/017068; No. 2003/0096980; No. 2002/0128218; No. 2002/0123476; U.S. Pat. No. 5,721,138.

The oligonucleotides of the present invention may be modified oligonucleotides. Unmodified nucleotide are often less optimal in some applications, e.g., prone to degradation bycellular nucleases. Chemical modifications to one or more of the subunits of oligonucleotide can confer improved properties, e.g., can render oligonucleotides more stable to nucleases. Typical oligonucleotide modifications are well-known in the art and may include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the modification or replacement of the 2′ hydroxyl on the ribose sugar; (iii) wholesale replacement of the phosphate moiety; (iv) modification or replacement of a naturally occurring base with a non-natural base; (v) replacement or modification of the ribose-phosphate backbone, e.g. with peptide nucleic acid (PNA); (vi) modification of the 3′ end or 5′ end of the oligonucleotide; and (vii) modification of the sugar, e.g., six membered rings. Oligonucleotides used in accordance with this invention can be synthesized by any number of means well-known in the art, or purchased from a variety of commercial vendors (LC Sciences, Houston, Tex.; Promega, Madison, Wis.; Invitrogen, Carlsbad, Calif.).

A wide variety of entities, e.g., ligands, can be coupled to the oligonucleotides as known in the art. Ligands can include naturally occurring molecules, or recombinant or synthetic molecules. Exemplary ligands include, but are not limited to, peptides, peptidomimetics, polylysine (PLL), polyethylene glycol (PEG), mPEG, cationic groups, spermine, spermidine, polyamine, thyrotropin, melanotropin, lectin, glycoprotein, surfactant protein A, mucin, glycosylated polyaminoacids, transferrin, aptamer, immunoglobulins (e.g., antibodies), insulin, transferrin, albumin, sugar, lipophilic molecules (e.g, steroids, bile acids, cholesterol, cholic acid, and fatty acids), vitamin A, vitamin E, vitamin K, vitamin B, folic acid, B12, riboflavin, biotin, pyridoxal, vitamin cofactors, lipopolysaccharide, hormones and hormone receptors, lectins, carbohydrates, multivalent carbohydrates, radiolabeled markers, fluoroscent dyes, and derivatives thereof. See., e.g., U.S. Pat. No. 6,153,737; U.S. Pat. No. 6,172,208; U.S. Pat. No. 6,300,319; U.S. Pat. No. 6,335,434; U.S. Pat. No. 6,335,437; U.S. Pat. No. 6,395,437; U.S. Pat. No. 6,444,806; U.S. Pat. No. 6,486,308; U.S. Pat. No. 6,525,031; U.S. Pat. No. 6,528,631; U.S. Pat. No. 6,559,279.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, the term “inhibit”, “down-regulate”, or “reduce”, means that the expression of the gene, or level of RNA molecules or equivalent RNA is reduced below that observed in the absence of modulator. The gene expression is down-regulated when expression of the gene, or level of RNA molecules or equivalent RNA molecules reduced at least 10% lower relative to a corresponding non-modulated control.

For in vivo delivery, the oligonucleotides can be formulated in liposomes. As used herein, a liposome is a structure having lipid-containing membranes enclosing an aqueous interior. Liposomes can have one or more lipid membranes. Liposomes with several nonconcentric membranes, i.e., several smaller vesicles contained within a larger vesicle, are termed multivesicular vesicles. Liposome compositions can be prepared by a variety of methods that are known in the art. See e.g., U.S. Pat. No. 5,171,678; U.S. Pat. No. 5,552,157; U.S. Pat. No. 5,565,213; U.S. Pat. No. 5,738,868; U.S. Pat. No. 5,795,587; U.S. Pat. No. 5,922,859; U.S. Pat. No. 6,077,663; WO 96/14057; WO 96/37194; Felgner et al., 8PNAS 7413-17 (1987); Behr, 5 Bioconj. Chem. 382-89 (1994); Lewis et al., 93 PNAS 3176-81 (1996).

As disclosed herein, the inventors have discovered that inhibitors of HOTAIR decrease cancer invasiveness and metastasis. Thus, in some embodiments, the present invention is directed to an inhibitor (e.g. antagonist) of HOTAIR lincRNA, which is expressed by the HOTAIR gene. In some embodiments, an inhibitor of HOTAIR is any agent which inhibits HOTAIR lincRNA function, or an inhibitor of HOTAIR gene expression to produce HOTAIR lincRNA. Any agent is encompassed for use, e.g. a small molecule inhibitor, gene silencing RNAi etc., are useful in the methods, compositions and kits as disclosed herein.

As used herein, the term “HOTAIR LincRNA” refers to the nucleic acid of SEQ ID NO: 1 as disclosed herein, and homologues thereof, including conservative substitutions, additions, deletions therein not adversely affecting the structure of function.

The nucleic acid sequence for human HOTAIR LincRNA transcript (SEQ ID NO: 1) is as follows:

| | |
|---|---|
| 1 | acattctgcc ctgatttccg gaacctggaa gcctaggcag gcagtgggga actctgactc |
| 61 | gcctgtgctc tggagcttga tccgaaagct tccacagtga ggactgctcc gtgggggtaa |
| 121 | gagagcacca ggcactgagg cctgggagtt ccacagacca acacccctgc tcctggcggc |
| 181 | tcccacccgg gacttagacc ctcaggtccc taatatcccg gaggtgctct caatcagaaa |
| 241 | ggtcctgctc cgcttcgcag tggaatggaa cggatttaga agcctgcagt aggggagtgg |
| 301 | ggagtggaga gagggagccc agagttacag acggcggcga gaggaaggag gggcgtcttt |
| 361 | atttttttaa ggccccaaag agtctgatgt ttacaagacc agaaatgcca cggccgcgtc |
| 421 | ctggcagaga aaaggctgaa atggaggacc ggcgccttcc ttataagtat gcacattggc |
| 481 | gagagaagtg ctgcaaccta aaccagcaat tacacccaag ctcgttgggg cctaagccag |
| 541 | taccgacctg gtagaaaaag caaccacgaa gctagagaga gagccagagg agggaagaga |
| 601 | gcgccagacg aaggtgaaag cgaaccacgc agagaaatgc aggcaaggga gcaaggcggc |
| 661 | agttcccgga acaaacgtgg cagagggcaa gacgggcact cacagacaga ggtttatgta |
| 721 | tttttatttt ttaaaatctg atttggtgtt ccatgaggaa aagggaaaat ctagggaacg |
| 781 | ggagtacaga gagaataatc cgggtcctag ctcgccacat gaacgcccag agaacgctgg |
| 841 | aaaaacctga gcgggtgccg gggcagcacc cggctcgggt cagccactgc cccacaccgg |
| 901 | gcccaccaag ccccgcccct cgcggccacc ggggcttcct tgctcttctt atcatctcca |
| 961 | tctttatgat gaggcttgtt aacaagacca gagagctggc caagcacctc tatctcagcc |
| 1021 | gcgcccgctc agccgagcag cggtcggtgg ggggactggg aggcgctaat taattgattc |
| 1081 | ctttggactg taaaatatgg cggcgtctac acggaaccca tggactcata aacaatatat |
| 1141 | ctgttgggcg tgagtgcact gtctctcaaa taatttttcc ataggcaaat gtcagagggt |
| 1201 | tctggatttt tagttgctaa ggaaagatcc aaatgggacc aattttagga ggcccaaaca |
| 1261 | gagtccgttc agtgtcagaa aatgcttccc caaaggggtt gggagtgtgt tttgttggaa |
| 1321 | aaaagcttgg gttataggaa agcctttccc tgctacttgt gtagacccag cccaatttaa |
| 1381 | gaattacaag gaagcgaagg ggttgtgtag gccggaagcc tctctgtccc ggctggatgc |
| 1441 | aggggacttg agctgctccg gaatttgaga ggaacataga agcaaaggtc cagcctttgc |
| 1501 | ttcgtgctga ttcctagact taagattcaa aaacaaattt ttaaaagtga aaccagccct |
| 1561 | agcctttgga agctcttgaa ggttcagcac ccacccagga atccacctgc ctgttacacg |
| 1621 | cctctccaag acacagtggc accgcttttc taactggcag cacagagcaa ctctataata |
| 1681 | tgcttatatt aggtctagaa gaatgcatct tgagacacat gggtaaccta attatataat |
| 1741 | gcttgttcca tacaggagtg attatgcagt gggaccctgc tgcaaacggg actttgcact |
| 1801 | ctaaatatag accccagctt gggacaaaag ttgcagtaga aaaatagaca taggagaaca |
| 1861 | cttaaataag tgatgcatgt agacacagaa ggggtattta aaagacagaa ataatagaag |
| 1921 | tacagaagaa cagaaaaaaa atcagcagat ggagattacc attcccaatg cctgaacttc |
| 1981 | ctcctgctat taagattgct agagaattgt gtcttaaaca gttcatgaac ccagaagaat |
| 2041 | gcaatttcaa tgtatttagt acacacacag tatgtatata aacacaactc acagaatata |
| 2101 | ttttccatac attgggtagg tatgcacttt gtgtatatat aataatgtat tttccatgca |
| 2161 | gttttaaaat gtagatatat taatatctgg atgcattttc tgtgcactgg ttttatatgc |
| 2221 | cttatggagt atatactcac atgtagctaa atagactcag gactgcacat tccttgtgta |
| 2281 | ggttgtgtgt gtgtggtggt tttatgcata aataaagttt tacatgtggt gaaaaaa |

Agents in General which Function as Inhibitors of HOTAIR

In some embodiments, the present invention relates to agents which inhibit HOTAIR LincRNA function or decrease HOTAIR LincRNA levels. In some embodiments, an inhibitor inhibits or decreases the expression of the HOTAIR gene to produce HOTAIR LincRNA. In some embodiments, inhibition is inhibition of the function of HOTAIR LincRNA. In alternative embodiments, inhibition can be inhibition of the HOTAIR gene, where inhibition of the HOTAIR gene will reduce or inhibit the production of HOTAIR LincRNA.

In some embodiments, inhibition of HOTAIR LincRNA and/or inhibition of the HOTAIR gene can be an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof. In some embodiments, agents useful in methods of the present invention include agents that function as inhibitors of the expression HOTAIR LincRNA from the HOTAIR gene.

Agents useful in the methods as disclosed herein can also inhibit the function of HOTAIR LincRNA and/or expression of HOTAIR LincRNA from the HOTAIR gene using “gene silencers”. Such “gene silencer” agents and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

As used herein, agents useful in the method as inhibitors of HOTAIR LincRNA function and/or inhibition of expression from the HOTAIR gene can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit HOTAIR LincRNA function and/or inhibit HOTAIR LincRNA expression from the HOTAIR gene. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof.

Alternatively, agents useful in the methods as disclosed herein as inhibitors of HOTAIR LincRNA function and/or inhibit HOTAIR LincRNA expression from the HOTAIR gene can be a chemicals, small molecule, large molecule or entity or moiety, including without limitation synthetic and naturally-occurring non-proteinaceous entities. In certain embodiments the agent is a small molecule having the chemical moieties as disclosed herein.

### Small Molecules

All of the applications set out in the above paragraphs are incorporated herein by reference. It is believed that any or all of the compounds disclosed in these documents are useful for treatment of metastatic cancers, including, for example, but are not limited to breast cancer. In some embodiments, one of ordinary skill in the art can use other agents as inhibitors of HOTAIR LincRNA function and/or inhibit HOTAIR LincRNA expression from the HOTAIR gene, for example antibodies, or RNAi are effective for the treatment or prevention of metastatic cancers as claimed herein. In some embodiments, agents inhibiting HOTAIR LincRNA function and/or inhibit HOTAIR LincRNA expression from the HOTAIR gene can be assessed in models to determine decrease in HOTAIR LincRNA levels in metastatic cancer as disclosed herein. For example, one can use an in vitro assay as disclosed in the Examples herein, where HOTAIR LincRNA level can be monitored in the presence and absence of inhibitors of HOTAIR LincRNA function and/or inhibit HOTAIR LincRNA expression from the HOTAIR gene by methods commonly known by persons in the art.

Nucleic Acid Inhibitors of HOTAIR LincRNA Function and/or Inhibit HOTAIR LincRNA Expression from the HOTAIR Gene.

In some embodiments, agents that inhibit HOTAIR LincRNA function and/or inhibit HOTAIR LincRNA expression from the HOTAIR gene are nucleic acids. Nucleic acid inhibitors of HOTAIR LincRNA function and/or the HOTAIR gene include, for example, but not are limited to, RNA interference-inducing molecules, for example but are not limited to siRNA, dsRNA, stRNA, shRNA and modified versions thereof, where the RNA interference molecule silences (e.g. “gene silences”) the function of HOTAIR LincRNA or the expression of HOTAIR LincRNA from the HOTAIR gene.

In some embodiments, HOTAIR LincRNA function and/or HOTAIR LincRNA expression from the HOTAIR gene can also be inhibited by “gene silencing” methods commonly known by persons of ordinary skill in the art. In some embodiments, a nucleic acid inhibitor of HOTAIR LincRNA function and/or its expression from the HOTAIR gene is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In some embodiments, a RNAi inhibitor of HOTAIR LincRNA can be any RNAi agent selected from siHOTAIR-1, 5′-GAACGGGAGUACAGAGAGAUU-3′; (SEQ ID NO: 34); siHOTAIR-2, 5′-CCACAUGAACGCCCAGAGAUU-3′; (SEQ ID NO: 35); and siHOTAIR-3, 5′-UAACAAGACCAGAGAGCUGUU-3′) (SEQ ID NO: 36).

The 5′ domain of HOTAIR LincRNA binds to PCR2 (see Tsai et al., Science, 2010, 329; 689), with the first 300 nucleotides of HOTAIR LincRNA reportedly bind to PRC2 (see Spitale et al., Epigenetics, 2011; 6; 539-543). Accordingly, in alternative embodiments, a RNAi inhibitor of HOTAIR LincRNA targets or bind within 1-300 nucleotides of HOTAIR LincRNA of SEQ ID NO: 1. In some embodiments, a RNAi inhibitor of HOTAIR LincRNA targets or bind within 1-100 nucleotides of HOTAIR LincRNA of SEQ ID NO:1, or a RNAi inhibitor targets 100-200 nucleotides of SEQ ID NO: 1 or targets a region between 200-300 nucleotides of SEQ ID NO: 1.

The 3′ domain of HOTAIR LincRNA binds to LSD1 (see Tsai et al., Science, 2010, 329; 689), with the most 3′ 600 nucleotides of HOTAIR LincRNA reportedly bin to LSD1 complex (see Spitale et al., Epigenetics, 2011; 6; 539-543). Accordingly, in some embodiments, a RNAi inhibitor of HOTAIR LincRNA targets the last 600 nucleotides (e.g., the most 3′ 600 nucleotides) of HOTAIR LincRNA of SEQ ID NO: 1, e.g., between nucleotides 1737-2337 of SEQ ID NO: 1. In some embodiments, a RNAi inhibitor of HOTAIR LincRNA targets anywhere within the nucleotides 1730-1830 of HOTAIR LincRNA of SEQ ID NO: 1, or anywhere within the nucleotides 1830-1930 of HOTAIR LincRNA of SEQ ID NO: 1, anywhere within the nucleotides 1930-2030 of HOTAIR LincRNA of SEQ ID NO: 1, anywhere within the nucleotides 2030-2130 of HOTAIR LincRNA of SEQ ID NO: 1, anywhere within the nucleotides 2230-2337 of HOTAIR LincRNA of SEQ ID NO: 1.

In some embodiments, a RNAi inhibitor of HOTAIR LincRNA for use in the methods and compositions as disclosed herein binds to a HOT site, as disclosed in Tsai et al., Science, 2010, 329; 689, which is incorporated herein in its entirety by reference. In some embodiments, a RNAi inhibitor of HOTAIR LincRNA binds to a HOT-S site 5′-AGGGACAG-3′ (SEQ ID NO: 38), or binds to a HOT-L site 5′-CCAGC-3′ (SEQ ID NO: 39) or 5′-CCAGG-3′ (SEQ ID NO: 40). In some embodiments, a RNAi inhibitor of HOTAIR LincRNA binds to a REST motif of 5′-ATGGACAGCGCC-3′ (SEQ ID NO: 41). In some embodiments, a RNAi inhibitor of HOTAIR LincRNA binds to a SUC12/LSD1 binding site in HOTAIR overexpression, e.g., a RNAi can bind to a region of the HOTAIR LincRNA comprising 5′-CCAGC-3′ (SEQ ID NO: 42) or 5′-CCAGG-3′ (SEQ ID NO: 43).

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. the β2-AR regulator gene sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target sequence.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting HOTAIR can be readily designed and tested. Accordingly, siRNAs useful for the methods described herein include siRNA molecules of about 15 to about 40 or about 15 to about 28 nucleotides in length, which are homologous to the β2-AR regulator gene. Preferably, the HOTAIR targeting siRNA molecules have a length of about 25 to about 29 nucleotides. More preferably, the HOTAIR targeting siRNA molecules have a length of about 27, 28, 29, or 30 nucleotides. The HOTAIR r gene targeting siRNA molecules can also comprise a 3′ hydroxyl group. The HOTAIR targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the HOTAIR LincRNA targeting RNA molecule, or a RNA targeting molecule targeting the HOTAIR gene has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment a HOTAIR targeting RNA molecule, e.g., a RNA molecule targeting the HOTAIR LincRNA and/or HOTAIR gene can be double stranded—one strand has a 3′ overhang and the other strand can be blunt-ended or have an overhang. In the embodiment in which HOTAIR targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Inhibition of HOTAIR LincRNA function as disclosed herein has been successfully targeted using siRNAs as disclosed herein. For example, gene silencing RNAi of HOTAIR LincRNA are commercially available, for example from Invitrogen. In some embodiments, gene silencing RNAi agents can be produced by one of ordinary skill in the art and according to the methods as disclosed herein. In some embodiments, the assessment of the knock down of a HOTAIR LincRNA levels and/or its inhibition from HOTAIR gene can be determined using commercially available kits known by persons of ordinary skill in the art. Others can be readily prepared by those of skill in the art based on the known sequence of the target mRNA.

In some embodiments, an inhibitor for use in the methods, compositions and kits disclosed herein is a gene silencing RNAi of HOTAIR LincRNA function and/or its expression from the HOTAIR gene, and in some embodiments, is a siRNA. In some embodiments, one can use any gene silencing siRNA which targets a region of the sequence of HOTAIR LincRNA with the sequence corresponding to SEQ ID NO: 1 as disclosed herein, to inhibit HOTAIR Linc RNA function or alternatively, in some embodiments, a silencing siRNA targets a region of the sequence of HOTAIR gene to prevent or inhibit the expression form the HOTAIR gene.

In some embodiments, siRNA sequences are chosen to maximize the uptake of the antisense (guide) strand of the siRNA into RISC and thereby maximize the ability of RISC to target HOTAIR LincRNA or the HOTAIR gene. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC.

In a preferred embodiment, the siRNA or modified siRNA, such as gene silencing RNAi agents, and/or gene activating RNAi agents are delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, HOTAIR LincRNA to inhibit its function and/or HOTAIR gene to inhibit the expression of HOTAIR LincRNA. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

In some embodiments, an agent is protein or polypeptide or RNAi agent which inhibits HOTAIR LincRNA function and/or its expression from the HOTAIR gene. In such embodiments cells can be modified (e.g., by homologous recombination) to provide increased expression of such an agent, for example by replacing, in whole or in part, the naturally occurring promoter with all or part of a heterologous promoter so that the cells express the inhibitor of HOTAIR LincRNA function and/or its expression from the HOTAIR gene, for example a protein or RNAi agent (e.g. gene silencing- or gene activating-RNAi agent). Typically, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the inhibitor agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of the peptide or nucleic acid agent inhibitor of HOTAIR LincRNA function and/or its expression from the HOTAIR gene can also include an affinity column containing agents which will bind to the protein; one or more column steps over such affinity resins as concanavalin A-agarose, Heparin-Toyopearl™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography.

In one embodiment, an inhibitor of HOTAIR LincRNA function and/or its expression from the HOTAIR gene can be obtained synthetically, for example, by chemically synthesizing a nucleic acid by any method of synthesis known to the skilled artisan. A synthesized nucleic acid inhibitor of HOTAIR LincRNA function and/or its expression from the HOTAIR gene can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability of a nucleic acid inhibitor is desired, nucleic acids having nucleic acid analogs and/or modified internucleoside linkages can be used. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro ‘phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5,714,606 to Acevedo, et al, 5,378,825 to Cook, et al., 5,672,697 and 5,466,786 to Buhr, et al., 5,777,092 to Cook, et al., 5,602,240 to De Mesmacker, et al., 5,610,289 to Cook, et al. and 5,858,988 to Wang, also describe nucleic acid analogs for enhanced nuclease stability and cellular uptake.

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

In some embodiments, an inhibitor of HOTAIR LincRNA function and/or its expression from the HOTAIR gene is a gene silencing siRNA molecule which beginning from about 25 to 50 nucleotides, from about 50 to 75 nucleotides, or from about 75 to 100 nucleotides downstream of the start of the HOTAIR LincRNA or the start codon of the HOTAIR gene. One method of designing a siRNA molecule of the present invention involves identifying the 29 nucleotide sequence motif AA(N29)TT (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but are not limited to the NCBI, BLAST, Derwent and GenSeq as well as commercially available oligosynthesis software such as Oligoengine®, can also be used to select siRNA sequences against EST libraries to ensure that only one gene is targeted.

In some embodiments, where the modulator is a gene activating RNAi agent, e.g. for upregulating latrophilin 2 expression or protein levels, a RNAi modulator agent can target nucleotide sequences can contain 5′ or 3′ UTRs and regions nearby the start codon.

Delivery of RNA Interfering Agents:

Methods of delivering RNAi agents, e.g., an siRNA, or vectors containing an RNAi agent, to the target cells (e.g., basal cells or cells of the lung ad/or respiratory system or other desired target cells) are well known to persons of ordinary skill in the art. In some embodiments, a RNAi agent inhibitor of HOTAIR LincRNA function and/or its expression from the HOTAIR gene can be administered to a subject via aerosol means, for example using a nebulizer and the like. In alternative embodiments, administration of a RNAi agent inhibitor of HOTAIR LincRNA function and/or its expression from the HOTAIR gene can include, for example (i) injection of a composition containing the RNA interfering agent, e.g., an siRNA, or (ii) directly contacting the cell, e.g., a cell of the respiratory system, with a composition comprising an RNAi agent, e.g., an siRNA. In another embodiment, RNAi agents, e.g., an siRNA can be injected directly into any blood vessel, such as vein, artery, venule or arteriole, via, e.g., hydrodynamic injection or catheterization. In some embodiments an RNAi inhibitor of HOTAIR LincRNA function and/or its expression from the HOTAIR gene can delivered to specific organs, for example the liver, bone marrow or systemic administration.

Administration can be by a single injection or by two or more injections. In some embodiments, a RNAi agent is delivered in a pharmaceutically acceptable carrier. A gene silencing-RNAi agent which inhibits HOTAIR LincRNA function and/or its expression from the HOTAIR gene can also be administered in combination with other pharmaceutical agents which are used to treat or prevent neurodegenerative diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen which is identified by the antibody. In some embodiments, the antibody can be any antibody which identifies an antigen expressed on cells expressing PCR2 proteins.

In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g. siRNAs (e.g. gene silencing-RNAi agents) which inhibits HOTAIR LincRNA function and/or its expression from the HOTAIR gene to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Alternatively, in other embodiments, a RNAi agent, e.g., a gene silencing- or gene activating RNAi agent which can also be introduced into cells via the vascular or extravascular circulation, the blood or lymph system, and the cerebrospinal fluid.

The dose of the particular RNAi agent will be in an amount necessary to effect RNA interference, e.g., gene silencing RNAi which inhibits HOTAIR LincRNA function and/or its expression from the HOTAIR gene thereby leading to reduction of HOTAIR LincRNA level.

It is also known that RNAi molecules do not have to match perfectly to their target sequence. Preferably, however, the 5′ and middle part of the antisense (guide) strand of the siRNA is perfectly complementary to the target nucleic acid sequence of HOTAIR LincRNA (SEQ ID NO: 1).

Accordingly, the RNAi molecules functioning as gene silencing-RNAi agents which inhibit HOTAIR LincRNA function and/or its expression from the HOTAIR gene as disclosed herein are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In some embodiments, a RNAi nucleic acid inhibitor of HOTAIR LincRNA inhibits its function or decreases HOTAIR LincRNA levels can be any agent which binds to and inhibits HOTAIR LincRNA. In some embodiments, a RNAi nucleic acid inhibitor of HOTAIR gene inhibits the expression of HOTAIR LincRNA from the HOTAIR gene can be any agent which binds to the HOTAIR gene and inhibits the expression of HOTAIR LincRNA.

In another embodiment of the invention, agents inhibiting HOTAIR LincRNA and/or its expression from the HOTAIR gene are catalytic nucleic acid constructs, such as, for example ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of HOTAIR can be achieved by techniques well known to those skilled in the art (for example Lleber and Strauss, (1995) Mol Cell Biol 15:540.551, the disclosure of which is incorporated herein by reference).

Subjects Amenable to Treatment with Inhibitors of HOTAIR LincRNA Function and/or its Expression from the HOTAIR Gene

In some embodiments, a cancer in which HOTAIR LincRNA function and/or its expression from the HOTAIR gene is inhibited by the methods and compositions as disclosed herein is a cancer which expresses cancer genes selected from the group of HER2/Her-2, BRAC1 and BRAC2, Rb, p53, and variants thereof.

In some embodiments of all aspects of the invention, the method are applicable to the treatment of any cancer in a subject, preferably a mammalian subject or human subject, where the metastatic cancer is for example, but not limited to mescenchymal in origin (sarcomas); fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant leukemias; lymphocytic leukemias; plasmacytomas; leiomyosarcomas; and rhabdomyosarcoma; cancers epithelial in origin (carcinomas); squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; transitional cell carcinomas; squamous cell carcinomas; choriocarcinomas; seminomas; embryonal carcinomas; malignant teratomas; and terato carcinomas; leukemia; acute lymphocytic leukemia and acute myelocytic leukemia (myeloblastic, promyeloblastic, myelomonocytic; monocytic, and erythroleukemia); chronic leukemia; chronic myelocytic (granulocytic) leukemia; chronic lymphocytic leukemia; polycythemia vera; lymphoma; Hodgkin's disease; non-Hodgekin's disease; multiple mycloma; Waldenström's macroglobulinemia; heavy chain disease. In some embodiments, the cancer is lymphia; leukemia; sarcoma; adenomas. In some embodiments, the cancer is acute lympoblastic leukemia (ALL).

In some embodiments, the metastatic cancer is breast cancer. In some embodiments, the cancer is metastatic breast cancer. In some embodiments, the breast cancer is primary breast cancer. In some embodiments, the cancer is lung cancer, and in some embodiments, the lung cancer is metastatic lung cancer. In some embodiments, the cancer is prostate cancer, or colon cancer, or hepatocellular carcinoma.

In some embodiments, examples of cancers that can be treated with inhibitors of HOTAIR include, for example but are not limited to, small or non-small cell lung, oat cell, papillary, bronchiolar, squamous cell, transitional cell, Walker), leukemia (e.g., B-cell, T-cell, HTLV, acute or chronic lymphocytic, mast cell, myeloid), histiocytoma, histiocytosis, Hodgkin disease, non-Hodgkin lymphoma, plasmacytoma, reticuloendotheliosis, adenoma, adenocarcinoma, adeno-fibroma, adenolymphoma, ameloblastoma, angiokeratoma, angiolymphoid hyperplasia with eosinophilia, sclerosing angioma, angiomatosis, apudoma, branchioma, malignant carcinoid syndrome, carcinoid heart disease, carcinosarcoma, colon cancer, prostate cancer, cementoma, cholan-gioma, cholesteatoma, chondrosarcoma, chondroblastoma, chondrosarcoma, chordoma, choristoma, craniopharyngioma, chrondroma, cylindroma, cystadenocar-cinoma, cystadenoma, cystosarcoma phyllodes, dysgerminoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendo-thelioma, hemangioma, hemangiopericytoma, hemangiosarcoma, hepatoma, hepatocellular cancer, islet cell tumor, Kaposi sarcoma, leiomyoma, leiomyosarcoma, leukosarcoma, Leydig cell tumor, lipoma, liposarcoma, lymphangioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuro-blastoma, neuroepithelioma, neurofibroma, neurofibromatosis, odontoma, osteoma, osteosarcoma, papilloma, paraganglioma, paraganglioma nonchromaffin, pinealoma, rhabdomyoma, rhabdomyosarcoma, Sertoli cell tumor, teratoma, theca cell tumor, and other diseases in which cells have become dysplastic, immortalized, or transformed.

Overexpression of HOTAIR LincRNA has been reported to predict tumor reoccurrence in hepatocellular carcinoma (see Yang et al., Ann Surg Oncol, 2011, 18; 1243-50, which is incorporated herein in its entirety by reference). Accordingly, the methods, systems and compositions as disclosed herein can be used to measure high levels of HOTAIR lincRNA to identify a subject with increase likelihood of cancer recurrence.

### Diagnosis Methods

The present invention also provides for the compositions and methods for using lincRNAs such as HOTAIR LincRNA as a biomarker for cancer prognosis. For example, a biological sample (e.g., a tumor sample) is obtained from a subject, then a lincRNA(s) (or downstream members of the gene set) is measured and compared with corresponding samples from normal subjects. Measuring methods include any method of nucleic acid detection, for example in situ hybridization for HOTAIR LincRNA using antisense DNA or cRNA oligonucleotide probes, ultra-high throughput sequencing, Nanostring technology, microarrays, rolling circle amplification, proximity-mediated ligation, PCR, qRT-PCR ChIP, ChIP-qPCR or antibodies, or protein or nucleic acid measurements of any of the several members that comprise PRC2 gene set. Comparatively high levels of HOTAIR LincRNA indicate metastasis or poor cancer prognosis. Similarly, comparatively low levels of JAM2, PCDH10 and PCDHB5 may be associated with high levels of HOTAIR and thus indicate cancer progression. HOTAIR LincRNA overexpression may also be identified by a shift in H3K27 patterns.

In particular, the inventors have demonstrated that a biological sample from a subject which has about at least a 125-fold higher expression of HOTAIR LincRNA as compared to the reference level (e.g., from a non-cancer biological sample) is indicative of the presence of metastatic cancer in the biological sample obtained from the subject. In some embodiments, where the level of HOTAIR LincRNA expression in the biological sample from the subject is at least about 200-fold or higher as compared to the HOTAIR LincRNA reference level (e.g., from a non-cancer biological sample) is indicative of the presence of metastatic cancer in the biological sample obtained from the subject.

### Assessing HOTAIR Expression in a Subject

As described herein, the inventors have identified that HOTAIR LincRNA levels, e.g., increased HOTAIR LincRNA levels above a normal reference level, e.g., increased by a statistically significant degree in tissue can be used for cancer prognosis. Accordingly, some embodiments of the invention are generally related to assays, methods and systems for determining HOTAIR LincRNA levels, or their downstream members, e.g., JAM3, PCDH10, PCDHB5 for assessing the extent or level of cancer in a subject. In certain embodiments, the assays, methods and systems relate to identifying a subject with cancer or a need for treatment for cancer. Certain embodiments of the invention are related to assays, methods and systems for identifying the severity of cancer in a sample, e.g., a biopsy sample, obtained from a subject. In certain embodiments, the assays, methods and systems are directed to determination of the expression level of a gene product (e.g. protein and/or gene transcript such as mRNA) in a biological sample of a subject. In certain embodiments the assays, methods, and systems are directed to determination of the level of HOTAIR LincRNA in a biological sample of a subject, where high levels of HOTAIR LincRNA in the subject identify the subject as likely to have cancer, and/or metastatic cancer. In some embodiments, where the level of HOTAIR LincRNA in the biological sample is at least about 125-fold increased as compared to a reference HOTAIR LincRNA level, the subject identified as likely to have cancer, and/or metastatic cancer. In some embodiments, where the level of HOTAIR LincRNA in the biological sample is at least about 200-fold increased as compared to a reference HOTAIR LincRNA level, the subject identified as likely to have cancer, and/or metastatic cancer. In some embodiments, where the level of HOTAIR LincRNA in the biological sample is between about 125-2000-fold higher as compared to a reference HOTAIR LincRNA level, the subject identified as likely to have cancer, and/or metastatic cancer. In such instances, a subject identified as likely to have cancer, and/or metastatic cancer can be treated with a more aggressive anti-cancer treatment regimen.

In some embodiments, where the level of HOTAIR LincRNA in the biological sample is at least about 200-fold increased as compared to a reference HOTAIR LincRNA level, the subject is predicted to have a poor outcome and low metastasis free survival, or a decreased survival chance as compared to a subject who has a HOTAIR LincRNA not statistically significant different or similar to reference HOTAIR LincRNA levels. In some embodiments, where the level of HOTAIR LincRNA in the biological sample is between about 125-2000-fold higher as compared to a reference HOTAIR LincRNA level, the subject is predicted to have a poor outcome and low metastasis free survival, or a decreased survival chance as compared to a subject who has a HOTAIR LincRNA not statistically significant different or similar to reference HOTAIR LincRNA levels. In such instances, a subject identified with a poor outcome and low metastasis free survival, or a decreased survival chance can be treated with a more aggressive anti-cancer treatment regimen.

In some embodiments, at least one or more other marker genes can be assessed, e.g., downregulated markers such as JAM3, PCDH10, PCDHB5, or upregulated markers such as ABL2, SNAIL, LAMB3 or LAMC2, i.e. at least two marker genes, or at least three marker genes, or at least four marker genes, or at least five marker genes, or at least six marker genes or more than 6 marker genes, where low expression levels of JAM3, PCDH10, PCDHB5 as compared to reference expression levels of JAM3, PCDH10, PCDHB5 identify the subject at risk of metastatic cancer, and high levels of ABL2, SNAIL, LAMB3 or LAMC2 as compared to reference levels for ABL2, SNAIL, LAMB3 or LAMC2 identify a subject at risk for metastatic cancer.

In some embodiments, where the level of JAM3 expression in the biological sample is at least about 50%, or about 60% or about 70% or about 80% decreased as compared to a reference JAM3 expression level, the subject identified as likely to have cancer, and/or metastatic cancer. In some embodiments, where the level of PCDH1 expression in the biological sample is at least about 50%, or about 60% or about 70% or about 80% decreased as compared to a reference PCDH1 expression level, the subject identified as likely to have cancer, and/or metastatic cancer. In some embodiments, where the level of PCDHB5 expression in the biological sample is at least about 55%, or about 60% or about 70% decreased as compared to a reference PCDHB5 expression level, the subject identified as likely to have cancer, and/or metastatic cancer. In such instances, a subject identified as likely to have cancer, and/or metastatic cancer can be treated with a more aggressive anti-cancer treatment regimen.

In some embodiments, where the level of ABL2 expression in the biological sample is at least about 2-fold, or about 3-fold or about 4-fold or about 5-fold or greater than 5-fold increased as compared to a reference ABL2 expression level, the subject identified as likely to have cancer, and/or metastatic cancer. In some embodiments, where the level of SNAIL expression in the biological sample is at least about 2-fold, or about 3-fold or greater than 3-fold increased as compared to a reference SNAIL expression level, the subject identified as likely to have cancer, and/or metastatic cancer. In some embodiments, where the level of LAMB3 expression in the biological sample is at least about 3-fold or about 4-fold or about 5-fold, or about 6-fold greater than 6-fold increased as compared to a reference LAMB3 expression level, the subject identified as likely to have cancer, and/or metastatic cancer. In some embodiments, where the level of LAMC2 expression in the biological sample is at least about 2-fold, or about 3-fold or about 4-fold or greater than 4-fold increased as compared to a reference LAMC2 expression level, the subject identified as likely to have cancer, and/or metastatic cancer. In such instances, a subject identified as likely to have cancer, and/or metastatic cancer can be treated with a more aggressive anti-cancer treatment regimen.

In certain embodiments, the subject may be exhibiting a sign or symptom of cancer. In certain embodiments, the subject may be asymptomatic or not exhibit a sign or symptom of cancer, but can be at risk of developing cancer due to certain risk factors as described herein.

In some embodiments, the methods and assays described herein include (a) transforming the biomarker product into a detectable gene target; (b) measuring the amount of the detectable gene target; and (c) comparing the amount of the detectable gene target to an amount of a reference, wherein if the amount of the detectable gene target (e.g., HOTAIR LincRNA) is statistically different from that of the amount of the reference level for the gene target (e.g., HOTAIR LincRNA), the subject is identified as having cancer or is in need of a treatment for cancer.

In some embodiments, the reference can be a level of HOTAIR LincRNA (e.g., HOTAIR LincRNA) expression of the biomarker in a normal healthy subject with no symptoms or signs of cancer or metastasis. For example, a normal healthy subject does not have cancer. In some embodiments, the reference can also be a level of expression of the biomarker (e.g., HOTAIR LincRNA) in a control sample, a pooled sample of control individuals or a numeric value or range of values based on the same. In some embodiments, the reference can also be a level of the biomarker in a tissue sample taken from non-cancerous tissue of the subject. In certain embodiments, wherein the progression of cancer in a subject is to be monitored over time, the reference can also be a level of biomarker (e.g., HOTAIR LincRNA) in a tissue sample taken from the tissue of the subject at an earlier date.

In certain embodiments, a biomarker (e.g., HOTAIR LincRNA) are upregulated in a biological sample, e.g., a biopsy sample from a subject with cancer. If the level of a biomarker (e.g., HOTAIR LincRNA) is higher than a reference level of that biomarker, the subject is more likely to have cancer or to be in need of a treatment for cancer. The level of a biomarker (e.g., HOTAIR LincRNA) which is higher than a reference level for that biomarker by at least about 10% than the reference amount, at least about 20%, at least about 30%, at least about 40%, at least about 50%, at least about 80%, at least about 100%, at least about 200%, at least about 300%, at least about 500% or at least about 1000% or more, is indicative that the subject has cancer. As discussed herein, in some embodiments, where the level of HOTAIR LincRNA in the biological sample is at least about 125-fold increased as compared to a reference HOTAIR LincRNA level, the subject identified as likely to have cancer, and/or metastatic cancer. In some embodiments, where the level of HOTAIR LincRNA in the biological sample is at least about 200-fold increased as compared to a reference HOTAIR LincRNA level, the subject identified as likely to have cancer, and/or metastatic cancer. In some embodiments, where the level of HOTAIR LincRNA in the biological sample is between about 125-2000-fold higher as compared to a reference HOTAIR LincRNA level, the subject identified as likely to have cancer, and/or metastatic cancer. In such instances, a subject identified as likely to have cancer, and/or metastatic cancer can be treated with a more aggressive anti-cancer treatment regimen.

In some embodiments, where the level of HOTAIR LincRNA in the biological sample is at least about 200-fold increased as compared to a reference HOTAIR LincRNA level, the subject is predicted to have a poor outcome and low metastasis free survival, or a decreased survival chance as compared to a subject who has a HOTAIR LincRNA level which is not statistically significant different or is similar to reference HOTAIR LincRNA level. In some embodiments, where the level of HOTAIR LincRNA in the biological sample is between about 125-2000-fold higher as compared to a reference HOTAIR LincRNA level, the subject is predicted to have a poor outcome and low metastasis free survival, or a decreased survival chance as compared to a subject who has a HOTAIR LincRNA which is not statistically significant different or is similar to reference HOTAIR LincRNA levels. In such instances, a subject identified with a poor outcome and low metastasis free survival, or a decreased survival chance can be treated with a more aggressive anti-cancer treatment regimen.

In certain embodiments marker genes (e.g., JAM, PCDH10, PCDHB5) are down-regulated in a biological sample, e.g., a biopsy sample from a subject with cancer. If the level of a gene expression product of a downregulated marker gene (e.g., JAM, PCDH10, PCDHB5) is lower than a reference level of that marker gene, the subject is more likely to have cancer or to be in need of a treatment for cancer. The level of a gene expression product of a downregulated marker gene (e.g., JAM, PCDH10, PCDHB5) which is lower than a reference level of that marker gene by at least about 10% than the reference amount, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100% is indicative that the subject has cancer. As discussed herein, in some embodiments, where the level of JAM3 expression in the biological sample is at least about 50%, or about 60% or about 70% or about 80% decreased as compared to a reference JAM3 expression level, the subject identified as likely to have cancer, and/or metastatic cancer. In some embodiments, where the level of PCDH1 expression in the biological sample is at least about 50%, or about 60% or about 70% or about 80% decreased as compared to a reference PCDH1 expression level, the subject identified as likely to have cancer, and/or metastatic cancer. In some embodiments, where the level of PCDHB5 expression in the biological sample is at least about 55%, or about 60% or about 70% decreased as compared to a reference PCDHB5 expression level, the subject identified as likely to have cancer, and/or metastatic cancer. In such instances, a subject identified as likely to have cancer, and/or metastatic cancer can be treated with a more aggressive anti-cancer treatment regimen.

In certain embodiments marker genes (e.g., ABL2, SNAIL, LAMB3 or LAMC2) are upregulated in a biological sample, e.g., a biopsy sample from a subject with cancer. If the level of a gene expression product of a downregulated marker gene (e.g., ABL2, SNAIL, LAMB3 or LAMC2) is higher than a reference level of that marker gene, the subject is more likely to have cancer or to be in need of a treatment for cancer. As discussed herein, in some embodiments, where the level of ABL2 expression in the biological sample is at least about 2-fold, or about 3-fold or about 4-fold or about 5-fold or greater than 5-fold increased as compared to a reference ABL2 expression level, the subject identified as likely to have cancer, and/or metastatic cancer. In some embodiments, where the level of SNAIL expression in the biological sample is at least about 2-fold, or about 3-fold or greater than 3-fold increased as compared to a reference SNAIL expression level, the subject identified as likely to have cancer, and/or metastatic cancer. In some embodiments, where the level of LAMB3 expression in the biological sample is at least about 3-fold or about 4-fold or about 5-fold, or about 6-fold greater than 6-fold increased as compared to a reference LAMB3 expression level, the subject identified as likely to have cancer, and/or metastatic cancer. In some embodiments, where the level of LAMC2 expression in the biological sample is at least about 2-fold, or about 3-fold or about 4-fold or greater than 4-fold increased as compared to a reference LAMC2 expression level, the subject identified as likely to have cancer, and/or metastatic cancer. In such instances, a subject identified as likely to have cancer, and/or metastatic cancer can be treated with a more aggressive anti-cancer treatment regimen.

In some embodiments, the biological sample used in the assay is a blood sample, or a urine sample, and in some embodiments, the biological sample is a biopsy sample.

### Methods for Measuring HOTAIR LincRNA Levels

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Methods to measure gene expression products associated with the marker genes described herein are well known to a skilled artisan. Such methods to measure gene expression products, e.g., protein level, include ELISA (enzyme linked immunosorbent assay), western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies can be made and/or are commercially available and can be used for the purposes of the invention to measure protein expression levels. Alternatively, since the amino acid sequences for the marker genes described herein are known and publicly available at NCBI website, one of skill in the art can raise their own antibodies against these proteins of interest for the purpose of the invention.

In another embodiment, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In certain embodiments, the gene expression products as described herein can be instead determined by determining the level of messenger RNA (mRNA) expression of genes associated with the marker genes described herein. Such molecules can be isolated, derived, or amplified from a biological sample, such as a lung biopsy. Detection of mRNA expression is known by persons skilled in the art, and comprise, for example but not limited to, PCR procedures, RT-PCR, Northern blot analysis, differential gene expression, RNA protection assay, microarray analysis, hybridization methods etc.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

Systems for Identifying a Subject with Cancer by Measuring HOTAIR Expression

In another embodiment of the assays described herein, the assay comprises or consists essentially of a system for transforming and measuring the amount of gene expression products of HOTAIR as described herein and comparing them to a reference expression level. If the comparison system, which can be a computer implemented system, indicates that the amount of the measured gene expression product is statistically different from that of the reference amount, the subject from which the sample is collected can be identified as having an increased risk for having cancer or for a subject in need of a treatment for cancer or metastasis.

Embodiments of the invention also provide for systems (and computer readable media for causing computer systems) to perform a method for identifying a subject with cancer by measuring the level of gene expression products of HOTAIR, or in some embodiments, other down-regulated markers (e.g., JAM, PCDH10, PCDHB5).

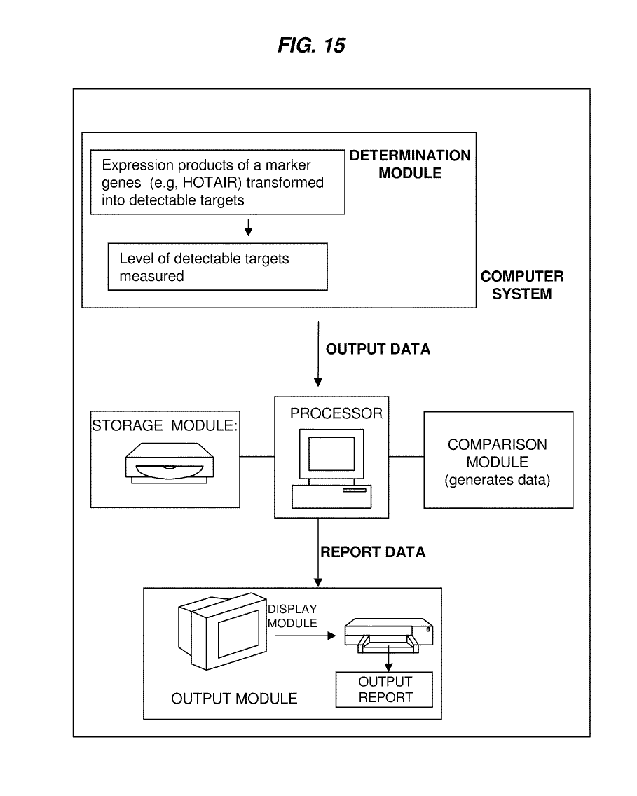

In one embodiment, provided herein is a system comprising: (a) at least one memory containing at least one computer program adapted to control the operation of the computer system to implement a method that includes (i) a determination module configured to identify and detect at the level of HOTAIR LincRNA in a biological sample obtained from a subject; (ii) a storage module configured to store output data from the determination module; (iii) a computing module adapted to identify from the output data whether the level of HOTAIR LincRNA measured in the biological sample obtained from a subject varies by a statistically significant amount from the HOTAIR LincRNA level found in a reference sample and (iv) a display module for displaying whether the level of HOTAIR LincRNA or other markers measured has a statistically significant variation in level in the biological sample obtained from a subject as compared to the reference HOTAIR LincRNA level and/or displaying the relative expression levels of the biomarkers, e.g., HOTAIR LincRNA levels and (b) at least one processor for executing the computer program (see FIG. 15).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination module, a storage module, a computing module, and a display module. The functional modules can be executed on one, or multiple, computers, or by using one, or multiple, computer networks. The determination module has computer executable instructions to provide e.g., levels of expression products etc in computer readable form.

The determination module can comprise any system for detecting a signal elicited from the marker genes described herein in a biological sample. In some embodiments, such systems can include an instrument, e.g., StepOnePlus Real-Time PCR systems (Applied Biosystems) as described herein for quantitative RT-PCR. In another embodiment, the determination module can comprise multiple units for different functions, such as amplification and hybridization. In one embodiment, the determination module can be configured to perform the quantitative RT-PCR methods described in the Examples, including amplification, detection, and analysis. In some embodiments, such systems can include an instrument, e.g., the Cell Biosciences NanoPro 1000 System (Cell Biosciences) for quantitative measurement of peptides and/or proteins.

In some embodiments, the determination module can be further configured to identify and detect the presence of at least one additional cancer-related marker gene.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as levels of the biomarkers or measured genes, e.g., HOTAIR LincRNA and other biomarker genes (e.g., JAM, PCDH10, PCDHB5) in subjects who do not have a symptom of cancer. In certain embodiments, the storage module stores the reference information such as expression levels of the marker genes (e.g., HOTAIR LincRNA, JAM, PCDH10, PCDHB5) described herein in a sample obtained from a healthy subject or in a sample from the subject taken at an earlier time.

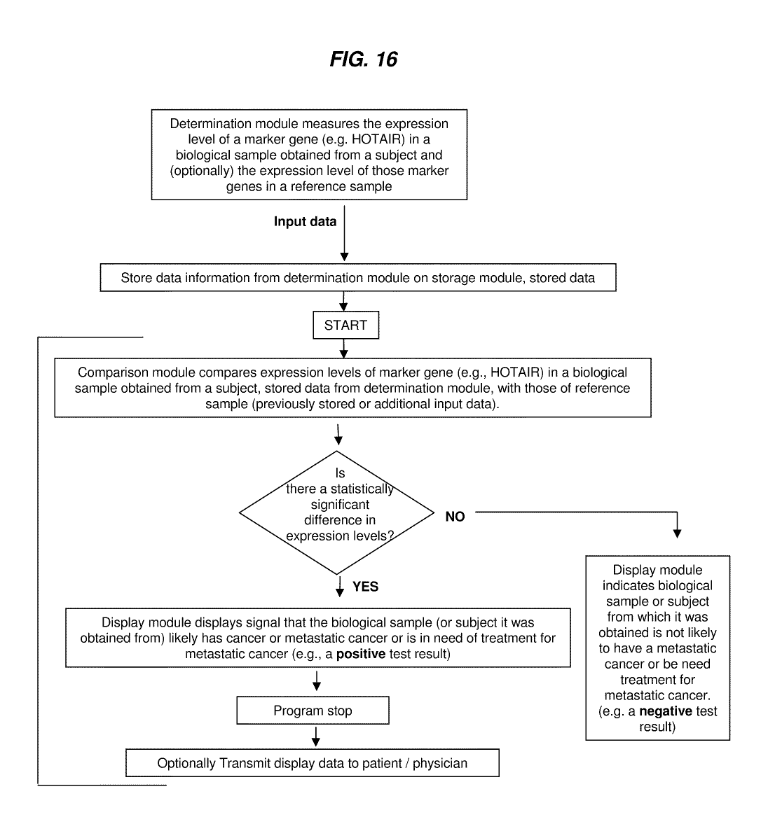

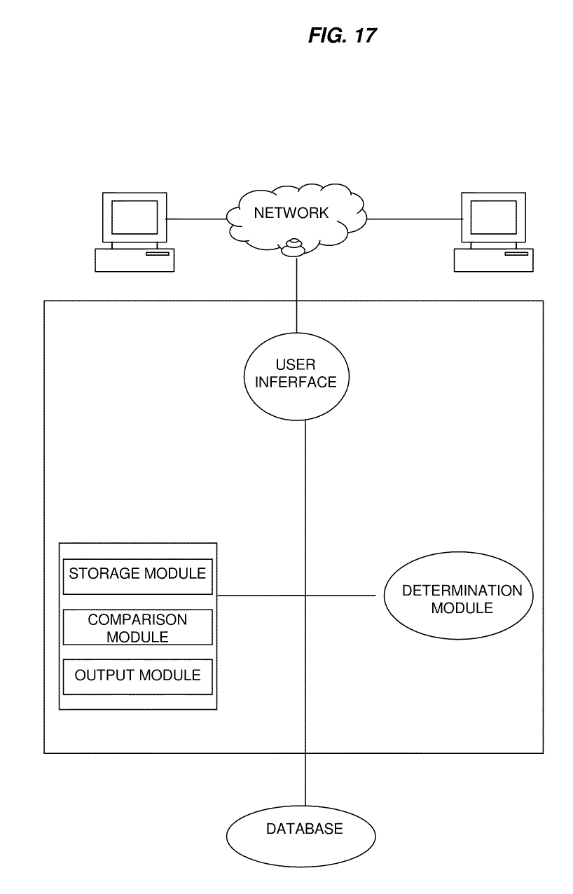

The “computing module” can use a variety of available software programs and formats for computing the relative expression level of the marker genes described herein. Such algorithms are well established in the art. A skilled artisan is readily able to determine the appropriate algorithms based on the size and quality of the sample and type of data. The data analysis tools described in Examples can be implemented in the computing module of the invention. In one embodiment, the computing module further comprises a comparison module, which compares the levels of the biomarkers (e.g., HOTAIR LincRNA, or PRC2 target genes, e.g., JAM, PCDH10, PCDHB5) in the biological sample obtained from a subject as described herein with the reference expression level of those marker genes (FIG. 16). By way of an example, when the level of HOTAIR LincRNA in a biological sample obtained from a subject is measured, a comparison module can compare or match the output data—with a reference HOTAIR LincRNA level in a reference sample. In certain embodiments, the reference expression level can have been pre-stored in the storage module. During the comparison or matching process, the comparison module can determine whether the expression level in the lung tissue sample obtained from a subject is lower than the reference expression level to a statistically significant degree. In various embodiments, the comparison module can be configured using existing commercially-available or freely-available software for comparison purpose, and may be optimized for particular data comparisons that are conducted.

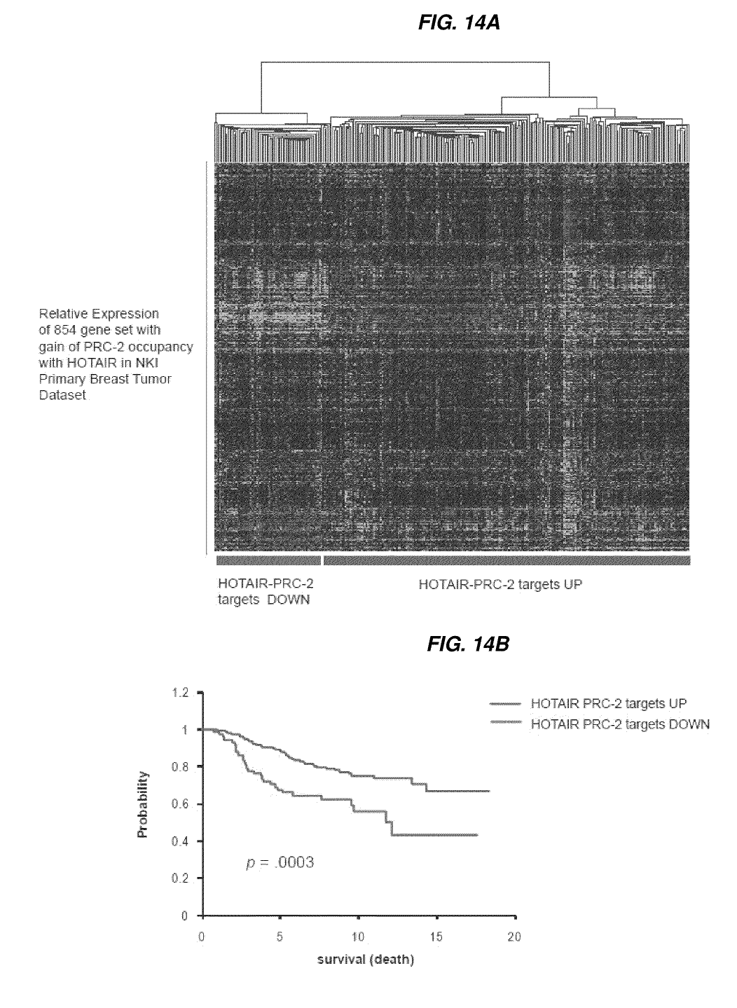

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers (FIG. 14).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be the relative levels of the biomarker genes (e.g., HOTAIR LincRNA, or PRC2 target genes, e.g., JAM, PCDH10, PCDHB5) in a biological sample obtained from a subject as compared to a reference expression level. In certain embodiments, the content displayed on the display module can indicate whether the marker genes measured have a statistically significant variation in expression (e.g., increase or decrease) between the biological sample obtained from a subject as compared to a reference expression level. In certain embodiments, the content displayed on the display module can indicate the degree to which the marker genes were found to have a statistically significant variation in expression between the biological sample obtained from a subject as compared to a reference expression level. In certain embodiments, the content displayed on the display module can indicate whether the subject has an increased risk of having cancer, and/or the severity of the cancer. In certain embodiments, the content displayed on the display module can indicate whether the subject is in need of a treatment for cancer. In certain embodiments, the content displayed on the display module can indicate whether the subject has an increased risk of having a more severe case of cancer or metastasis. In some embodiments, the content displayed on the display module can be a numerical value indicating one of these risk or probabilities. In such embodiments, the probability can be expressed in percentages or a fraction. For example, higher percentage or a fraction closer to 1 indicates a higher likelihood of a subject having cancer or metastasis. In some embodiments, the content displayed on the display module can be single word or phrases to qualitatively indicate a risk or probability. For example, a word “unlikely” can be used to indicate a lower risk for having cancer, while “likely” can be used to indicate a high risk for having cancer.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for assessing the state of the lungs of subject by measuring the expression level of at least two of the marker genes described herein, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

Biological Sample

Provided herein are methods, assays and systems for assessing a subject with cancer by measuring the level of at least one marker gene as described herein (e.g., HOTAIR LincRNA, or PRC2 target genes, e.g., JAM, PCDH10, PCDHB5) in a biological sample obtained from a subject.

The term “biological sample” as used herein denotes a sample taken or isolated from a biological organism, e.g., tumor biopsy sample, tissue cell culture supernatant, cell lysate, a homogenate of a tissue sample from a subject or a fluid sample from a subject. Exemplary biological samples include, but are not limited to, biopsies, the external sections of the tumor, lung epithelial cells, etc. The term also includes both a mixture of the above-mentioned samples. The term “biological sample” also includes untreated or pretreated (or pre-processed) biological samples. A biological sample obtained from a subject can contain cells from subject, but the term can also refer to non-cellular biological material, such as non-cellular fractions that can be used to measure gene expression levels. In some embodiments, the biological sample can be from a resection, biopsy, or core needle biopsy. In addition, fine needle aspirate samples can be used. Samples can be either paraffin-embedded or frozen tissue.

In some embodiments, a biological sample can be obtained by removing a sample of cells from a subject, but can also be accomplished by using previously isolated cells (e.g. isolated at a prior timepoint and isolated by the same or another person). In addition, a biological sample can be freshly collected or a previously collected sample. Furthermore, a biological sample can be utilized for the detection of the presence and/or quantitative level of a gene expression product of marker genes as described herein. In some embodiments, a maker gene expression product is a biomolecule. Representative biomolecules include, but are not limited to, DNA, RNA, mRNA, polypeptides, and derivatives and fragments thereof. In some embodiments, a biological sample can be used for expression analysis for diagnosis of a disease or a disorder, e.g., cancer or metastasis, using the methods, assays and systems of the invention.

In some embodiments, a biological sample is a biological fluid. Examples of biological fluids include, but are not limited to, saliva, blood, sputum, an aspirate, and any combinations thereof. In some embodiments, a biological sample is an untreated tissue sample. As used herein, the phrase “untreated tissue sample” refers to a tissue sample that has not had any prior sample pre-treatment except for dilution and/or suspension in a solution. Exemplary methods for treating a tissue sample include, but are not limited to, centrifugation, filtration, sonication, homogenization, heating, freezing and thawing, and any combinations thereof. In some embodiments, a biological sample is a frozen lung tissue sample, e.g., a frozen tissue or fluid sample such as sputum. A frozen biological sample can be thawed before employing methods, assays and systems of the invention. After thawing, a frozen sample can be centrifuged before being subjected to methods, assays and systems of the invention.

In some embodiments, a biological sample can be treated with at least one chemical reagent, such as a protease inhibitor. In some embodiments, a biological sample is a clarified tissue sample, for example, by centrifugation and collection of a supernatant comprising the clarified lung tissue sample. In some embodiments, a biological sample is a pre-processed tissue sample, for example, supernatant or filtrate resulting from a treatment selected from the group consisting of centrifugation, filtration, sonication, homogenization, lysis, thawing, amplification, purification, restriction enzyme digestion ligation and any combinations thereof. In some embodiments, a biological sample can be a nucleic acid product amplified after polymerase chain reaction (PCR). The term “nucleic acid” used herein refers to DNA, RNA, or mRNA.

In some embodiments, a biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

The skilled artisan is well aware of methods and processes appropriate for pre-processing of biological samples required for determination of expression of gene expression products as described herein.

### Assays for Identifying Compounds Useful to Inhibit HOTAIR

As described herein, the inventors have identified that HOTAIR LincRNA is upregulated and PRC2 target genes (e.g., JAM2, PCDH10, PCDHB5) are downregulated to a statistically significant degree in biological sample obtained from a subject with cancer or metastasis. Accordingly, some embodiments of the invention are generally related to assays, methods and systems for assessing whether a compound can be useful in treating or preventing the progression of cancer by identifying compounds that inhibit HOTAIR LincRNA upregulation (e.g., by inhibiting HOTAIR gene) or inhibit HOTAIR LincRNA function, or alternatively, inhibiting the downstream effects of high levels of HOTAIR LincRNA, e.g, downregulation or suppression of PRC2 target genes (e.g., JAM2, PCDH10, PCDHB5).

In certain embodiments, the assays, methods and systems relate to identifying a compound which decreases HOTAIR LincRNA levels in a biological sample with high levels of HOTAIR LincRNA. In certain embodiments, the assays, methods and systems are directed to determination of the level of HOTAIR LincRNA in a biological sample which has been treated with a compound.

In some embodiments, the methods and assays described herein comprise contacting a biological sample with an test agent, and (a) transforming the biomarker (e.g., HOTAIR LincRNA) into a detectable gene target; (b) measuring the amount of the detectable gene target (e.g., HOTAIR LincRNA); and (c) comparing the amount of the detectable gene target (e.g., HOTAIR LincRNA) to an amount of a reference level of HOTAIR LincRNA, wherein if the amount of the detectable HOTAIR LincRNA is decreased by a statistically different from that of the amount of the reference level (e.g., in the absence of a test agent, or negative control test agent), the compound is identified as being useful in decreasing the levels HOTAIR LincRNA and can be useful the treatment of cancer or metastasis.

In alternative embodiments, the methods and assays described herein comprise contacting a biological sample with an test agent, and (a) transforming the gene expression product of a downregulated marker gene, e.g., a PRC2 target genes (e.g., JAM2, PCDH10, PCDHB5) into a detectable gene target; (b) measuring the amount of the detectable gene target (e.g., JAM2 and/or PCDH10 and/or PCDHB5); and (c) comparing the amount of the detectable gene target (e.g., JAM2 and/or PCDH10 and/or PCDHB5) to an amount of a reference level of JAM2 and/or PCDH10 and/or PCDHB5, wherein if the amount of the detectable JAM2 and/or PCDH10 and/or PCDHB5 is increased by a statistically different from that of the amount of the reference level (e.g., in the absence of a test agent, or negative control test agent), the compound is identified as being useful in increasing a PRC2 target gene (e.g., JAM2, PCDH10, PCDHB5) and can be useful the treatment of cancer or metastasis.

In some embodiments, the methods for measuring gene expression are as described elsewhere herein. In some embodiments, there are provided systems for performing the assays described herein. Examples of such systems are described in detail elsewhere herein.

Contacting a Biological Sample with a Compound

Provided herein, assays, methods and systems for assessing whether a compound can be useful in treating or preventing cancer by identifying an agent which inhibits HOTAIR LincRNA, and/or increases the expression of PRC2 target gene (e.g., JAM2, PCDH10, PCDHB5). In some embodiments, these aspects of the invention involve contacting a biological sample with a compound or agent. A biological sample can be contacted with a compound at any time prior to transforming the gene expression product into a detectable gene target. For example, the biological sample can be contacted with the compound 1 minute prior to transformation, 30 minutes prior to transformation, 1 hour prior to transformation, 12 hours prior to transformation, 1 day prior to transformation, 1 week prior to transformation, 1 month prior to transformation, or more.

In some embodiments, a biological sample can be contacted with a compound once or multiple times. In some embodiments, a biological sample can be contacted with a compound repeatedly. In some embodiments, a biological sample can be contacted with a combination of two or more compounds. In some embodiments, one or more compounds can be compounds which have not been identified as useful in treating cancer or metastasis and one or more compounds can be compounds which have previously been identified as useful or used to treat cancer or metastasis.

In some embodiments, a subject can be contacted with a compound and a biological sample is subsequently obtained from the subject for use in a assay, method, or system as described herein. In some embodiments, a biological sample is obtained from a subject and subsequently contacted with a compound. In some embodiments, the biological sample contacted with a compound is not obtained directly from a subject, e.g. the biological sample comprises cultured cells.

In some embodiments, a compound can be any agent as that term is defined herein, and in some embodiments, can be a hormone, enzyme, cell, gene silencing molecule, inhibitor of an enzyme, small molecule, peptide, protein, nucleotide, antibody, antibody fragment, growth factor, virus, and/or bacterium.

In summary, the cancer transcriptome is more complex than previously believed. In addition to protein coding genes and microRNAs, dysregulated expression of lincRNAs is likely pervasive in human cancers and can drive cancer development and progression. Notably, the lincRNA HOTAIR can act as a significant regulator of metastatic progression. HOTAIR recruits PRC2 complex to specific targets genes genome-wide, leading to H3K27 trimethylation and epigenetic silencing of metastasis suppressor genes (FIG. 4F). The inventors discoveries substantially expand on the known scope of lincRNA action, showing that a single lincRNA can act like a transcription factor and affect hundreds of target genes genome-wide. The concept of epigenomic reprogramming by lincRNAs may also be applicable to many other human disease states where lincRNAs are misexpressed or chromatin states are aberrantly specified. HOTAIR is normally involved in specifying the chromatin state associated with fibroblasts from anatomically posterior and distal sites, and upon its ectopic expression in cancer, can re-impose that chromatin state in a manner reminiscent of homeosis—where one body segment is transformed into another by misexpression of HOX genes (Krumlauf, 78 Cell 191-201 (1994)). Because HOTAIR LincRNA is not expressed at many body sites to which breast cancer metastasize (e.g., the lung), HOTAIR LincRNA expression is less likely a homotypic homing system for cells. Rather, the inventors demonstrate that HOTAIR enables a developmental state associated with gene expression programs that are conducive to cell motility and matrix invasion, properties that cancer cells can commandeer.

Tailoring adjuvant therapy in breast cancer patients relies on prognostic and predictive factors, most of which are currently established by histopathological analysis of tumors. Currently, prognostic factors include tumor size, lymph node status, tumor grade, HER2 status, and lymphovascular invasion. Predictive factors include estrogen and progesterone receptors expression, HER2 overexpression or amplification. The quality of these assessments are an essential prerequisite for an optimal therapeutic decision. If the prognostic and predictive values of multigenes signatures are confirmed by on-going clinical studies, this approach could enter the clinical practice in the coming years and result in improved accuracy of adjuvant therapies in breast cancer patients. See, e.g., Fiche et al., 3(119) Rev. Med. Suisse, 1737-42 (2007).

The interdependence between HOTAIR LincRNA and PRC2 has potential therapeutic implications. High levels of HOTAIR LincRNA identifies tumors that are especially dependent on PRC2 activity and therefore sensitive to small molecules inhibitors of PRC2 (Tan et al., 21 Genes Devel. 1050-63 (2007)). Conversely, tumors that overexpress Polycomb proteins may be sensitive to therapeutic strategies that target endogenous HOTAIR LincRNA or inhibit HOTAIR-PRC2 interactions, providing new avenues in cancer therapy. HOTAIR LincRNA is a powerful marker for predicting which patients are most likely to have cancer that will metastasize, identifying patients that may require more aggressing treatment.

### Kits

Another aspect of the present invention relates to a nucleic acid construct of HOTAIR LincRNA comprising SEQ ID NO: 1 or a fragment of at least 20 bp thereof. Another aspect of the present invention relates to an expression vector comprising a nucleic acid construct of HOTAIR LincRNA of SEQ ID NO: 1 or a fragment of at least 20 bp of SEQ ID NO: 1 for expressing HOTAIR LincRNA. Any expression vector known to one of ordinary skill in the art is encompassed for use in the present invention.

### EXAMPLES

### Materials and Methods

Human material was obtained from Johns Hopkins Hospital and the Netherlands Cancer Institute. Expression of HOX transcripts was determined using ultra-high-density HOX tiling arrays7 and qRT-PCR. Kaplan-Meier analyses of breast cancer patients were as described¹³. We used retroviral transduction to overexpress HOTAIR LincRNA and luciferase, and used siRNA or shRNA to deplete the indicated transcripts. Matrix invasion was measured by the transwell Matrigel assay. The inventors implanted cells in the mammary fat pad of severe combined immunodeficient (SCID) mice, and monitored primary tumour growth and lung metastasis by bioluminescence. Cells were injected into the tail vein of nude mice, and lungs were analysed at 9 weeks to quantify lung colonization in vivo. ChIP-chip was performed as described⁷ using human whole genome promoter tiling arrays (Roche Nimblegen). Module map and Gene Ontology enrichment analyses were done using Genomica²⁰.

Reagents. The MDA-MB-231, SK-BR-3, MCF-10A, MCF-7, HCC1954, T47D and MDA-MB-453 cell lines were obtained from the American Type Culture Collection (ATCC). The H16N2 cell line was a gift from V. Band. pLZRS, pLZRS-luciferase and pSuper Retro-shGFP, -shSUZ12 and -shEZH2 (ref. 25) were obtained from P. Khavari. pLZRS-HOTAIR and pLZRS-EZH2-Flag were constructed by subcloning the full-length human HOTAIR7 or Flag-EZH2-ER fusion protein (representing amino acids 1-751 of EZH2 fused with the murine oestrogen receptor (amino acids 281-599)) into pLZRS using theGateway cloning system (Invitrogen).

Human materials. Normal breast organoid RNA was prepared as reported26. In brief, tissues from reduction mammoplasties performed at Johns Hopkins Hospital were mechanically macerated then digested overnight with hyaluronic acid and collagenase. The terminal ductal units are placed into suspension by this method; they were then isolated by serial filtration. Samples were treated with TRIzol and RNA extracted. Fresh frozen primary breast tumour specimens were obtained from the Department of Pathology breast tumour bank; specimens were all from patients 45-55 years of age, with oestrogen receptor expression by immunohistochemistry as performed during routine tumour staging at diagnosis, for uniformity of samples.

Metastatic breast carcinoma samples were obtained from the Rapid Autopsy Program at Johns Hopkins Hospital²⁷. All specimens were snap-frozen at time of autopsy and stored at 280 uC. Twenty 20-mm sections were obtained from metastasis to the liver (for uniformity of samples) and embedded in OCT. These slices were macerated by use of the BioMasher centrifugal sample preparation device (Cartagen), with 350 ml of lysis buffer from the Qiagen RNeasy Mini Extraction kit. RNA extraction was completed with the flow-through from the BioMasher, as per the commercial protocol.

HOTAIR LincRNA expression and survival/metastasis analysis of primary breast tumours. The database of 295 breast cancer patients from the Netherlands Cancer Institute with detailed clinical and gene expression data was used¹³. Clinical data are available at world wide web at: “microarray-pubs.stanford.edu/wound_NKI”, http://www.rii.com/publications, or http://microarrays.nki.nl. RNA from 132 primary breast tumours from the NKI 295 cohort was isolated along with RNA from normal breast organoid cultures (n56). HOTAIR LincRNA and GAPDH were measured by qRT-PCR. HOTAIR LincRNA values were normalized to GAPDH and expressed relative to pooled normal HOTAIR RNA levels. For both univariate and multivariate analysis, the expression of HOTAIR LincRNA was treated as a binary variable divided into ‘high’ and ‘low’ HOTAIR LincRNA expression. To determine the criteria for high HOTAIR expression, the minimum relative level of HOTAIR LincRNA seen in six metastatic breast cancer samples (see FIG. 1c and accompanying methods) was determined ($125 above normal). By this criteria, 44 primary breast tumours were categorized as high, and 88 were labelled as low, out of 132 tumours. For statistical analysis, overall survival was defined by death from any cause. Distant metastasis free probability was defined by a distant metastasis as the first recurrence event.

Kaplan-Meier survival curves were compared by the Cox-Mantel log-rank test in Winstat (R. Fitch software). Multivariate analysis by the Cox proportional hazard method was done using SPSS 15.0 (SPSS)

RNA expression analysis. qRT-PCR: total RNA from cells was extracted using TRIzol and the RNeasy mini kit (Qiagen). RNA levels (starting with 50-100 ng per reaction) for a specific gene (primer set sequences listed in Supplementary Table 4) were measured using the Brilliant SYBR Green II qRT-PCR kit (Strategene) according to manufacturer instructions. All samples were normalized to GAPDH.

HOX tiling array: RNA samples (primary or metastatic breast carcinoma in channel in Cy5 channel and normal breast organoid RNA representing a pool of six unique samples in Cy3 channel) were labelled and hybridized to a custom human HOX tiling array with 50-base-pair resolution (Roche Nimblegen) as described. For each sample, robust multichip average (RMA) normalized intensity values for previously defined peaks encoding HOX-coding-gene exons (as defined in version HG17) and HOX lincRNAs (as defined previously⁷) were determined relative to normal. Unsupervised hierarchical clustering was performed by CLUSTER²⁸.

Microarray: total RNA from cells was extracted using TRIzol and the RNeasy mini kit (Qiagen) and hybridized to Stanford human oligonucleotide (HEEBO) arrays as described²⁹. Data analysis was done using CLUSTER²⁸. Gene transfer experiments. Retrovirus was generated using amphotrophic phoenix cells and used to infect target cells as described³⁰. For LZRS vector, HOTAIR, EZH2-ER, and firefly luciferase, no further selection was done after infection. For pRetro-Super-shGFP, -shSUZ12 and -shEZH2, target cells were selected using puromycin (0.5 μg ml⁻¹). Many of the epigenetic changes due to HOTAIR expression were only seen after several cell passages; thus all experiments post-HOTAIR transduction were done after passage 10.

Non-radioactive in situ hybridization of paraffin sections. Digoxigenin (DIG)-labelled sense and antisense RNA probes were generated by PCR amplification of T7 promoter incorporated into the primers. In vitro transcription was performed with DIG RNA labelling kit and T7 polymerase according to the manufacturer's protocol (Roche Diagnostics). Sections (5-mm thick) were cut from the paraffin blocks, deparaffinized in xylene, and hydrated in graded concentrations of ethanol for 5 min each. Sections were incubated with 1% hydrogen peroxide, followed by digestion in 10 μg ml⁻¹ proteinase K at 37° C. for 30 min. Sections were hybridized overnight at 55° C. with either sense or antisense riboprobes at 200 ng ml⁻¹ dilution in mRNA hybridization buffer (Chemicon). The next day, sections were washed in 2×SSC and incubated with 1:35 dilution of RNase A cocktail (Ambion) in 2×SSC for 30 min at 37° C. Next, sections were stringently washed twice in 2×SSC/50% formamide, followed by one wash in 0.08×SSC at 55° C. Biotin-blocking reagents (Dako) were applied to the section to block the endogenous biotin. For signal amplification, a horseradish peroxidase (HRP)-conjugated sheep anti-DIG antibody (Roche) was used to catalyse the deposition of biotinyl-tyramide, followed by secondary streptavidin complex (GenPoint kit; Dako). The final signal was developed with DAB (GenPoint kit; Dako), and the tissues were counterstained in haematoxylin for 30 s.

RNA interference. RNA interference for HOTAIR was done as described. In brief, cells were transfected with 50 nM siRNAs targeting HOTAIR (siHOTAIR-1, 5′-GAACGGGAGUACAGAGAGAUU-3′ (SEQ ID NO 34); siHOTAIR-2, 5′-CCACAUGAACGC CCAGAGAUU-3′ (SEQ ID NO: 35); siHOTAIR-3, 5′-UAACAAGACCAGAGAGCUGUU-3′ (SEQ ID NO: 36) or siGFP (5′-CUACAACAGCCACAACGUCdTdT-3′ (SEQ ID NO:37) using Lipofectamine 2000 (Invitrogen) as per the manufacturer's direction. Total RNA was collected 72 h later for qRT-PCR analysis. RNA interference of EZH2 and SUZ12 was done by infecting target cells with retrovirus expressing shEZH2, shSUZ12 and shGFP as described²⁵. To confirm knockdown, protein lysates were resolved on 10% SDS-PAGE followed by immunoblot analysis as described³⁰ using anti-SUZ12 (Abcam), anti-EZH2 (Upstate) and anti-tubulin (Santa Cruz).

Matrigel invasion assay and cell proliferation assay. The matrigel invasion assay was done using the Biocoat Matrigel Invasion Chamber from Becton Dickson according to manufacturer protocol. In brief, 5×10⁴ cells were plated in the upper chamber in serum-free media. The bottom chamber contained DMEM media with 10% FBS. After 24-48 h, the bottom of the chamber insert was fixed and stained with Diff-Quick stain. Cells on the stained membrane were counted under a dissecting microscope. Each membrane was divided into four quadrants and an average from all four quadrants was calculated. Each matrigel invasion assay was at least done in biological triplicates. For invasion assays in the H16N2 cell line using EZH2-ER, all experiments (both vector and with EZH2-ER) were done in the presence of 500 nM oestradiol.

For cell proliferation assays, 1×10³ cells were plated in quadruplicate in 96-well plates and cell number was calculated using the MTT assay (Roche).

Soft agar colony formation assay. Soft agar assays were constructed in 6-well plates. The base layer of each well consisted of 2 ml with final concentrations of 13 media (RPMI (HCC1954), McCoy's Media (SKBR3), or DMEM (MDAMB-231) plus 10% or 2% heat-inactivated FBS (Invitrogen)) and 0.6% low melting point agarose. Plates were chilled at 4° C. until solid. Upon this, a 1-ml growth agar layer was poured, consisting of 1×10⁴ cells (infected with either LZRS-HOTAIR or LZRS vector as described earlier) suspended in 1× media and 0.3% low melting point agarose. Plates were again chilled at 4° C. until the growth layer congealed. A further 1 ml of 1× media without agarose was added on top of the growth layer on day 0 and again on day 14 of growth. Cells were allowed to grow at 37° C. for 1 month and total colonies were counted (>200 μm in diameter for MDA-MB-231; >50 μm diameter for HCC1954 and SKBR3). Assays were repeated a total of three times. Results were statistically analysed by paired t-test using the PRISM Graphpad program.

Mammary fat pad xenografts. Six-week-old female SCID beige mice were purchased from Charles River laboratories, housed at the animal care facility at Stanford University Medical Center and kept under standard temperature, humidity and timed lighting conditions and provided mouse chow and water ad libitum. MDA-MB-231-Luc or MDA-MB-231-Luc tumour cells transduced with HOTAIR were injected directly into the mammary fat pad of the mice semi-orthotopically (n=10 each) in 0.05 ml of sterile DMEM (2,500,000 cells per animal).

Mouse tail-vein assay. Female athymic nude mice were used. Two-million MDA-MB-231 HOTAIR-luciferase or vector-luciferase cells in 0.2 ml PBS were injected by the tail vein into individual mice (18 for each cell line). Mice were observed generally for signs of illness weekly for the length of the experiment. The lungs were excised and weighed fresh, then bisected. Half was fixed in formalin overnight then embedded in paraffin, from which sections were made and stained with haematoxylin and eosin by our pathology consultation service. These slides were examined for the presence of micrometastases, which were counted in three low-power (35) fields per specimen. The other half of the tumour was fast-frozen into OCT and stored at −80° C. RNA was extracted by the TRIzol protocol from ten sections, 20-μm thick each, obtained from the frozen sections. RT-PCR confirmed expression of HOTAIR LincRNA in lungs bearing micrometastases of MDA-MB-231 HOTAIR cells at the end of the experiment.

Bioluminescence imaging. Mice received luciferin (300 mg kg⁻¹, 10 min before imaging) and were anaesthetized (3% isoflurane) and imaged in an IVIS spectrum imaging system (Xenogen, part of Caliper Life Sciences). Images were analysed with Living Image software (Xenogen, part of Caliper Life Sciences). Bioluminescent flux (photons s⁻¹ sr⁻¹ cm⁻²) was determined for the primary tumours or lungs (upper abdomen region of interest).

ChIP-chip. ChIP-chip experiments were done as previously described. Each experiment was done in biological triplicate. The following antibodies were used: anti-H3K27me3 (Abcam), anti-SUZ12 (Abcam) and anti-EZH2 (Upstate). Immunoprecipitated DNA was amplified using the Whole Genome Amplification kit (Sigma) based on the manufacturer's protocol. Amplified and labelled DNA was hybridized to the HG18 whole genome two array promoter set from Roche Nimblegen. Probe labelling, hybridization, and data extraction and analysis were performed using Roche Nimblegen protocols. The relative ratio of HOTAIR to vector was calculated for each promoter peak by extracting the normalized (over input) intensity values for promoter peaks showing peaks with an FDR score≦0.2 in either vector or HOTAIR cells. These values were weighted to determine the significance of the relative ratio: using Cluster28, only those promoters with a consistent relative ratio (HOTAIR/vector) ≧1.5-fold or ≦0.5-fold in two out of the three ChIP were selected and displayed in TreeView. Selected ChIP-chip results were confirmed by PCR using the Lightcycler 480 SYBR Green I kit.

TaqMan real-time PCR assays. A panel of 96 TaqMan real-time PCR HOX assays was developed targeting 43 HOX lincRNAs and 39 HOX transcription factors across the four HOX loci. Two housekeeping genes (ACTB and PPIA) were also included in this panel in triplicates as endogenous controls for normalization between samples. The transcript specificity and genome specificity of all TaqMan assays were verified using a position specific alignment matrix to predict potential cross-reactivity between designed assays and genome-wide non-target transcripts or genomic sequences. Using this HOX assay panel we profiled 88 total RNA samples from a cohort of five normal breast organoids, 78 primary breast tumours (from the NKI 295 cohort) and five metastatic breast tumours. cDNAs were generated from 30 ng total RNA using the High Capacity cDNA Reverse Transcription Kit (Life Technologies). The resulting cDNA was subjected to a 14-cycle PCR amplification followed by realtime PCR reaction using the manufacturer's TaqMan PreAmp Master Mix Kit Protocol (Life Technologies). Four replicates were run for each gene for each sample in a 384-well format plate on a 7900HT Fast Real-Time PCR System (Life Technologies). Between the two measured endogenous control genes (PPIA and ACTB), we chose PPIA for normalization across different samples based on the fact that this gene showed the most relatively constant expression in different breast carcinomas (data not shown).

Gene set analysis. For gene set enrichment analysis, gene sets from fifteen different H3K27, SUZ12 or EZH2 global occupancy lists from the indicated cell lineages were procured. Pattern matching between the 854-gene set with increased PRC2 occupancy and these 15 gene sets were visualized using CLUSTER and TreeView. The significance of enrichment between these gene sets was calculated using module map analysis implemented in Genomica20 (corrected for multiple hypotheses using FDR)

### Example 1

### In Vitro and In Vivo Examination of HOTAIR

Human materials were obtained from the frozen tumor bank and the Rapid Autopsy Program of the Department of Surgical Pathology, Johns Hopkins Hospital, and the Netherlands Cancer Institute. The expression of HOX coding and lincRNAs in human breast cancer samples was determined using a custom ultra-high density HOX tiling array. Rinn et al., 2007. HOTAIR was quantified by qRT-PCR. Survival and metastasis analysis was done using the Netherlands Cancer Institute cohort of breast cancer patients with stage I/II disease (van de Vijver et al., 2002) using standard statistical methods. HOTAIR LincRNA was introduced into cells by retroviral transduction; gene depletion was accomplished using siRNA or shRNA targeting the transcript. Matrix invasion was measured by the transwell MATRIGEL™ matrix assay. Cells were injected into the tail vein of nude mice, and lungs were analyzed histologically at 9 weeks to determine lung metastasis in vivo. Chromatin immunoprecipitation micro array analysis was performed using ChIP-chip analysis, as described (Rinn et al., 2007), on human whole genome promoter tiling arrays (Roche Nimblegen, Inc., Madison, Wis.). Module map and GO enrichment analyses were done using the Genomica genomic data analysis and visualization tool (Segal et al., 2004).

### Example 2

### Unique Association of HOTAIR with Patient Outcome

To determine whether the expression of other HOX lincRNAs in addition to HOTAIR can predict patient outcome, the inventors measured the expression levels of 43 different HOX lincRNAs and all 39 HOX coding genes in 78 primary breast tumors from the NKI 295 breast cancer patient cohort. Results confirm the widespread dysregulation of HOX lincRNAs in breast cancer (FIG. 6). Results from our tiling array had identified a subset of genes that showed a distinct set of HOX coding genes and lincRNAs, including HOTAIR, that are variably overexpressed in primary tumors and frequently overexpressed in metastatic samples (FIG. 1b). This large data set of qRT-PCR expression of multiple HOX coding and lincRNAs was utilized to determine if other transcripts highlighted in FIG. 1b [including HOXC10, HOXC1 1, HOXC13, and nc-HOXC10-124 (shown by EST mapping to also comprise transcripts labeled nc-HOXC10-126A and nc-HOXC10-127A)] were linked to patient outcome. For each transcript, patients with high versus low expression showed no statistically significant difference in overall survival or metastasis free survival (Table 1, FIG. 7).

TABLE 1
Lack of association between other HOX transcripts with patient outcome

| | Death | Metastasis |
|---|---|---|
| | P value | P value |
| High HOXC10 | 0.192 | 0.114 |
| High HOXC11 | 0.582 | 0.325 |
| High HOXC13 | 0.853 | 0.972 |
| High HOXC10-124 | 0.487 | 0.161 |

^{a}RNA expression data (as measured by qRT-PCR) from seventy-eight primary breast tumors used to determine association. ncHOXC10-1 24, −126A, −126B are believed to represent exons of one lincRNA and are represented by ncHOXC1 0-124 here.

TABLE 2
Multivariate analysis of risk factors for death and
metastasis as the first recurrence event in early breast cancer

| | Death | | Metastasis | |
|---|---|---|---|---|
| | Hazard | | Hazard | |
| | Ratio | P value | Ratio | P value |
| High HOTAIR expression^{a} | 3.313 | 0.001 | 3.468 | 0.001 |
| Age | 0.754 | 0.468 | 0.745 | 0.374 |
| Diameter of tumor, per cm | 0.651 | 1.184 | 1.524 | 0.175 |
| Lymph node status, | 3.125 | 0.033 | 3.348 | 0.014 |
| per positive node | | | | |
| Tumor grade | 2.161 | 0.600 | 1.668 | 0.133 |
| Vascular invasion | 1.935 | 0.001 | 1.726 | 0.001 |
| Estrogen receptor status, | 0.329 | 0.020 | 0.695 | 0.422 |
| positive vs. negative | | | | |
| No adjuvant therapy vs. chemo or | 1.824 | 0.290 | 1.376 | 0.498 |
| hormonal therapy | | | | |

^{a}Modeled as a binary variable with High HOTAIR expression defined as primary breast tumors with relative HOTAIR expression ≧125 fold above normal (representing the minimum level of expression seen in a panel of metastatic tumors). Using this criteria, High HOTAIR expression represents 44/132 primary breast tumors surveyed.

TABLE 3
PRC-2 ChIP mapping data procured for gene set analysis

| Gene Set | Species | Platform | Description |
|---|---|---|---|
| SUZ12_Prostate Cancer | Human | Avia System Biology | SUZ12 occupancy PC-3 and |
| Cell Line¹ | | hu6K promoter set | LNCaP cell lines |
| EZH2_Prostate Cancer | Human | Custom 20K | EZH2 transcriptional targets |
| Cell Line¹ | | cDNA microarray | RWPE prostate cell line |
| PRC_2 Prostate Cancer² | Human | Agilent human | PRC-2 occupancy Metastatic |
| | | proximal promoter set | prostate cancer tissue |
| SUZ12_Colon Cancer | Human | Nimblegen-Roche | SUZ12 occupancy SW480 colon |
| Cell Line³ | | 5K promoter set | carcinoma line |
| SUZ12_Breast Cancer | Human | Nimblegen-Roche | SUZ12 occupancy MCF-7 breast |
| Cell Line³ | | 5K promoter set | carcinoma line |
| PRC-2_Embryonic | Human | Nimblegen-Roche | PRC-2 occupancy embryonic |
| Fibroblast Cell Line⁴ | | promoter tiliing array | Lung TIG3 line |
| H3K27_Lung | Human | Nimblegen-Roche | H3K27 occupancy neonatal |
| Fibroblast Cells⁵ | | 5K promoter set | primary lung fibroblasts |
| SUZ12_Foreskin | Human | Nimblegen-Roche | SUZ12 occupancy neonatal |
| Fibroblast Cells^{a} | | HG18 two array set | primary foreskin fibroblasts |
| H3K27_Foreskin | Human | Niimblegen-Roche | H3K27 occupancy neonatal |
| Fibroblast Cells⁵ | | 5K promoter set | primary foreskin fibroblasts |
| H3K27_Embryonic | Human | Agilent Whole | H3K27 occupancy WA09 |
| Stem Cell⁶ | | Genome Array | embryonic stem cells |
| SUZ12_Embryonic | Human | Agilent Whole | SUZ12 occupancy WA09 |
| Stem Cell⁶ | | Genome Array | embryonic stem cells |
| PRC-2_Embryonic | Human | Agilent Whole | PRC-2 occupancy WA09 |
| Stem Cell⁶ | | Genome Array | embryonic stem cells |
| SUZ12_Embryonic | Mouse | Agilent Mouse | SUZ12 occupancy mouse |
| Stem Cell⁷ | | Promoter Array Set | embryonic stem cells |
| SUZ12_Embryonic | Mouse | Nimblegen-Roche | SUZ12 occupancy mouse |
| Stem Cell³ | | 1.5 kb promoter set | embryonic stem cells |

¹Yu et al., 12 Canc. Cell 419 (2007);
²Yu et al., 67 Canc. Res.10657 (2007);
³Squazzo et al., 16 Genome Res. 890 (2006);
⁴Bracken et al., 20 Genes & Devel. 1123 (2006);
⁵O'Geen et al., 3 PLoS Gen. e89 (2007);
⁶Lee et al., 125 Cell 301 (2006);
⁷Boyer et al., 441 Nature 349 (2006);
^{a}Current Study

### Example 3

### A HOTAIR-PRC-2 Gene Set Signature can Predict Patient Outcome

To determine if the 854 gene set representing promoters with an increase in PRC-2 occupancy upon HOTAIR LincRNA overexpression (FIG. 3A) can be used as a diagnostic “fingerprint” for patient outcome, the gene expression of these 854 genes was extracted from the microarray data set of all 295 primary breast tumors from the NKI 295 patient cohort. Unsupervised hierarchical clustering of these data revealed a subset of patients that showed a distinct relative down-regulation of genes from the larger gene set (FIG. 14). Patients showing this unique signature was predictive for overall survival (p=0.0003).

TABLE 4
PCR primer pairs for qRT-PCR

| Gene Name | Forward | Reverse |
|---|---|---|
| HOTAIR | GGTAGAAAAAGCAACCACGAAGC | ACATAAACCTCTGTCTGTGAGTGCC |
| | (SEQ ID NO: 4) | (SEQ ID NO: 5) |
| GAPDH | CCGGGAAACTGTGGCGTGATGG | AGGTGGAGGAGTGGGTGTCGCTGTT |
| | (SEQ ID NO: 6) | (SEQ ID NO: 7) |
| LAMB3 | GCCACATTCTCTACTCGGTGA | CCAAGCCTGAGACCTACTGC |
| | (SEQ ID NO: 8) | (SEQ ID NO: 9) |
| SNAIL | TGACCTGTCTGCAAATGCTC | CAGACCCTGGTTGCTTCAA |
| | (SEQ ID NO: 10) | (SEQ ID NO: 11) |
| LAMC2 | CTCTGCTTCTCGCTCCTCC | TCTGTGAAGTTCCCGATCAA |
| | (SEQ ID NO: 12) | (SEQ ID NO: 13) |
| ABL2 | GGACACTTCACTTTGCTGCC | TAGTGCCTGGGGTTCAACAT |
| | (SEQ ID NO: 14) | (SEQ ID NO: 15) |
| JAM2 | TCTTTTGGGGCAGAAAAC | AAGATGGCGAGGAGG |
| | (SEQ ID NO: 16) | (SEQ ID NO: 17) |
| PCDH10 | CCCGTCTACACTGTGTCCCT | GGAGTACACGACCTCACCGT |
| | (SEQ ID NO: 18) | (SEQ ID NO: 19) |
| PCDHB5 | AGGTGTGTTTGACCGGAGAC | TCCCTATTTCTTCACCAGCG |
| | (SEQ ID NO: 20) | (SEQ ID NO: 21) |

TABLE 5
PCR primer pairs for ChIP verification

| Gene Name | Forward | Reverse |
|---|---|---|
| JAM2 | ACCTGACTTCCAGCACGAGT | CCAACTCCTTTCTTCCCCTC |
| | (SEQ ID NO: 22) | (SEQ ID NO: 23) |
| HOXD10 | GCTGAGGCGCTTTAATGAAC | GGTCCCAGAAACTCTGACCA |
| | (SEQ ID NO: 24) | (SEQ ID NO: 25) |
| PR | TCTCCAACTTCTGTCCGAGG | CACGAGTTTGATGCCAGAGA |
| | (SEQ ID NO: 26) | (SEQ ID NO: 27) |
| EphA1 | ATATGACAAACACGGCCCAT | GGTGGTTAACTTGGGGAACA |
| | (SEQ ID NO: 28) | (SEQ ID NO: 29) |
| PCDH10 | ACCAGGCTCTGTTCTGTTCG | TCTTGGGTCATAGGGGTCTG |
| | (SEQ ID NO: 30) | (SEQ ID NO: 31) |
| PCDHB5 | AGACCGGCAATTTGCTTCTA | TCTGGGGCATGGTCATTTAT |
| | (SEQ ID NO: 32) | (SEQ ID NO: 33) |

### Example 4

### RNAi of HOTAIR

RNA interference for HOTAIR was done as described (Rinn et al., 2007). Briefly, cells were transfected with 50 nM siRNAs targeting HOTAIR LincRNA:

| (SEQ ID NO: 34) | |
|---|---|
| siHOTAIR-1, | 5′-GAACGGGAGUACAGAGAGAUU-3′; |
| (SEQ ID NO: 35) | |
| siHOTAIR-2, | 5′-CCACAUGAACGCCCAGAGAUU-3′; |
| (SEQ ID NO: 36) | |
| siHOTAIR-3, | 5′-UAACAAGACCAGAGAGCUGUU-3′); |
| or | |
| (SEQ ID NO: 37) | |
| siGFP, | 5′-CUACAACAGCCACAACGUCdTdT-3′ |

using LIPOFECTAMINE™ 2000 transfection reagent (Invitrogen, Carlsbad, Calif.) as per the manufacturer's direction. Total RNA was collected 72 hours later for qRT-PCR analysis (FIG. 10).

### REFERENCES

All references are incorporated in their entirety by reference
1. Amaral, P. et al., The eukaryotic genome as an RNA machine Science 319, 1787-1789 (2008).
2. The FANTOM Consortium. The transcriptional landscape of the mammalian genome. Science 309, 1559-1563 (2005).
3. Guttman, M. et al. Chromatin signature reveals over a thousand highly conserved large non-coding RNAs in mammals. Nature 458, 223-227 (2009).
4. Calin, G. A. et al. Ultraconserved regions encoding ncRNAs are altered in human leukemias and carcinomas. Cancer Cell 12, 215-229 (2007).
5. Yu, W. et al. Epigenetic silencing of tumour suppressor gene p15 by its antisense RNA. Nature 451, 202-206 (2008).
6. Ponting, C. et al., Evolution and functions of long noncoding RNAs. Cell 136, 629-641 (2009).
7. Rinn, J. L. et al. Functional demarcation of active and silent chromatin domains in human HOX loci by noncoding RNAs. Cell 129, 1311-1323 (2007).
8. Khalil, A. M. et al. Many human large intergenic noncoding RNAs associate with chromatin-modifying complexes and affect gene expression. Proc. Natl. Acad. Sci. USA 106, 11667-11672 (2009).
9. Raman, V. et al. Compromised HOXA5 function can limit p53 expression in human breast tumours. Nature 405, 974-978 (2000).
10. Wu, X. et al. HOXB7, a homeodomain protein, is overexpressed in breast cancer and confers epithelial-mesenchymal transition. Cancer Res. 66, 9527-9534 (2006).
11. Sparmann, A. et al., Polycomb silencers control cell fate, development and cancer. Natl. Rev. 6, 846-856 (2006).
12. Kleer, C. G. et al. EZH2 is a marker of aggressive breast cancer and promotes neoplastic transformation of breast epithelial cells. Proc. Natl. Acad. Sci. USA 100, 11606-11611 (2003).
13. van de Vijver, M. J. et al. A gene-expression signature as a predictor of survival in breast cancer. N. Engl. J. Med. 347, 1999-2009 (2002).
14. Ma, L., Teruya-Feldstein, J. & Weinberg, R. A. Tumour invasion and metastasis initiated by microRNA-10b in breast cancer. Nature 449, 682-688 (2007).
15. Novak, P. et al. Agglomerative epigenetic aberrations are a common event in human breast cancer. Cancer Res. 68, 8616-8625 (2008).
16. Naik, M. U., et al., Attenuation of junctional adhesion molecule-A is a contributing factor for breast cancer cell invasion. Cancer Res. 68, 2194-2203 (2008).
17. Fox, B. P. et al., Invasiveness of breast carcinoma cells and transcript profile: Eph receptors and ephrin ligands as molecular markers of potential diagnostic and prognostic application. Biochem. Biophys. Res. Commun 318, 882-892 (2004).
18. Herath, N. I., et al., Epigenetic silencing of EphA1 expression in colorectal cancer is correlated with poor survival. Br. J. Cancer 100, 1095-1102 (2009).
19. The Gene Ontology Consortium. Gene ontology: tool for the unification of biology. Nature Genet. 25, 25-29 (2000).
20. Segal, E., et al., A module map showing conditional activity of expression modules in cancer. Nature Genet. 36, 1090-1098 (2004).
21. Srinivasan, D. & Plattner, R. Activation of Abl tyrosine kinases promotes invasion of aggressive breast cancer cells. Cancer Res. 66, 5648-5655 (2006).
22. Olmeda, D. et al. SNAI1 is required for tumor growth and lymph node metastasis of human breast carcinoma MDA-MB-231 cells. Cancer Res. 67, 11721-11731 (2007).
23. Marinkovich, M. P. Tumour microenvironment: laminin 332 in squamous-cell carcinoma. Natl. Rev. 7, 370-380 (2007).
24. Tan, J. et al. Pharmacologic disruption of Polycomb-repressive complex 2-mediated gene repression selectively
25. Sen, G. L., et al., A. Control of differentiation in a self-renewing mammalian tissue by the histone demethylase JMJD3. Genes Dev. 22, 1865-1870 (2008).
26. Bergstraesser, L. M. & Weitzman, S. A. Culture of normal and malignant primary human mammary epithelial cells in a physiological manner simulates in vivo growth patterns and allows discrimination of cell type. Cancer Res. 53, 2644-2654 (1993).
27. Wu, J. M. et al. Heterogeneity of breast cancer metastases: comparison of therapeutic target expression and promoter methylation between primary tumors and their multifocal metastases. Clin. Cancer Res. 14, 1938-1946 (2008).
28. Eisen, M. B., et al., Cluster analysis and display of genome-wide expression patterns. Proc. Natl. Acad. Sci. USA 95, 14863-14868 (1998).
29. Rinn, J. L., et al., Anatomic demarcation by positional variation in fibroblast gene expression programs. PLoS Genet. 2, e119 (2006).

We claim:

1. A method for the treatment of metastatic cancer in a subject comprising administering to a subject having metastatic cancer an effective amount of a RNAi inhibitor of HOTAIR lincRNA levels or function, or an effective amount of a RNAi inhibitor to inhibit HOTAIR LincRNA expression from the HOTAIR gene.

2. The method of claim 1, wherein the presence of metastatic cancer in the subject is indicated by high levels of HOTAIR lincRNA expression.

3. The method of claim 1, wherein the RNAi inhibitor of HOTAIR lincRNA is selected from the group consisting of siRNA, miRNA, stRNA, snRNA, and antisense nucleic acid.

4. A method of decreasing HOTAIR lincRNA level in a cancer cell, comprising contacting the cancer cell with a RNAi inhibitor of HOTAIR lincRNA levels or function, or an effective amount of a RNAi inhibitor to inhibit HOTAIR LincRNA expression from the HOTAIR gene.

5. The method of claim 4, wherein the RNAi inhibitor of HOTAIR lincRNA is selected from the group consisting of siRNA, miRNA, stRNA, snRNA, and antisense nucleic acid.

6. The method of claim 5, wherein the nucleic acid inhibitor is a siRNA.

7. A method for detecting a metastatic cancer in a subject, comprising;
contacting a biological sample from the subject with at least one nucleic acid binding probe to measure the level of HOTAIR lincRNA expression in the biological sample;
comparing the level of HOTAIR lincRNA expression in the biological sample to a reference level of HOTAIR lincRNA expression from a biological sample from a healthy population,
wherein an increased level of HOTAIR lincRNA expression in the biological sample from the subject compared to the reference level of HOTAIR lincRNA expression indicates likelihood of the subject having a metastatic cancer.

8. The method of claim 7, wherein a subject identified to have likelihood of having a metastic cancer is administered a RNAi inhibitor of HOTAIR lincRNA levels or function, or an effective amount of a RNAi inhibitor to inhibit HOTAIR LincRNA expression from the HOTAIR gene in an effective amount to inhibit cancer metastasis in the subject.

9. The method of claim 7, wherein a subject identified to have likelihood of having a metastic cancer is administered an agent which inhibits the function of PRC2 in an effective amount to inhibit cancer metastasis in the subject.

10. The method of claim 9, wherein the agent which inhibits the function PRC2 is a small molecule inhibitor of PRC2.

11. The method of claim 9, wherein the agent which inhibits the function PRC2 is an agent which inhibits the interaction of HOTAIR lincRNA with PRC2.

12. The method of claim 1, wherein the metastatic cancer is breast cancer.

13. The method of claim 4, wherein the metastatic cancer is breast cancer.

14. The method of claim 7, wherein the metastatic cancer is breast cancer.

15. An assay for measuring the level of HOTAIR lincRNA in a biological sample from a subject; comprising at least one agent that specifically binds to HOTAIR lincRNA, wherein binding of the agent to HOTAIR lincRNA results in a detectable signal.

16. The assay of claim 15, wherein the assay is a device comprising the assay and comprises at least a solid support wherein the agent that specifically binds to HOTAIR lincRNA is deposited on the support.

17. The assay of claim 16, wherein the solid support is in the format of a dipstick, a test strip, a latex bead, a microsphere, or a multi-well plate.

18. The assay of claim 15 comprising;
a measuring assembly yielding a detectable signal from an assay indicating the level of HOTAIR lincRNA from a biological sample from a subject; and
an output assembly for displaying an output content for the user.

19. The method of claim 4, wherein decreasing HOTAIR lincRNA level in a cancer cell inhibits the invasiveness of a cancer cell that expresses high levels of HOTAIR LincRNA.

20. A method of modulating histone H3 lysine 27 methylation in a cell comprising contacting that cell with a RNAi inhibitor of HOTAIR lincRNA levels or function, or an effective amount of a RNAi inhibitor to inhibit HOTAIR LincRNA expression from the HOTAIR gene.

21. The assay of claim 18, wherein the assay is automated.
